(19) United States
(12) Patent Application Publication
(10) Pub. No.: US 2026/0109970 A1
(43) Pub. Date: Apr. 23, 2026

(54) MAGNETIC HYDROGEL PARTICLES AND METHODS OF USE

(71) Applicant: The Regents of the University of California, Oakland, CA (US)

(72) Inventors: Brian PAEGEL, Irvine, CA (US); Valerie CAVETT, Irvine, CA (US)

(21) Appl. No.: 19/284,375

(22) Filed: Jul. 29, 2025

Related U.S. Application Data
(63) Continuation of application No. PCT/US2024/013545, filed on Jan. 30, 2024.
(60) Provisional application No. 63/442,396, filed on Jan. 31, 2023.
(60) Provisional application No. 63/504,191, filed on May 24, 2023.

(51) Int. Cl. C12N 15/10

(52) U.S. Cl. CPC C12N 15/1068

(57) ABSTRACT

The present invention relates to particles comprising a magnetic core coated in a polymeric gel, methods of making such particles, and methods of using such particles, for example to support both chemical and biological synthesis.

### CROSS-REFERENCE TO RELATED APPLICATIONS

This application is a continuation of International Application No. PCT/US2024/013545, filed Jan. 30, 2024, which claims priority to U.S. Provisional Application Ser. No. 63/442,396, filed Jan. 31, 2023, and U.S. Provisional Application Ser. No. 63/504,191, filed May 24, 2023, which are hereby incorporated herein by reference in their entirety.

### STATEMENT REGARDING FEDERAL RIGHTS

This invention was made with Government support under Grant No. R35GM140890, awarded by the National Institutes of Health. The Government has certain rights in the invention.

### REFERENCE TO AN ELECTRONIC SEQUENCE LISTING

The contents of the electronic sequence listing (146392063501 seqlist.xml; Size: 25,652 bytes; and Date of Creation: Jul. 22, 2025) is herein incorporated by reference in its.

### FIELD OF THE INVENTION

The present invention relates to particles comprising a magnetic core coated in a polymeric gel, methods of making such particles, and methods of using such particles, for example to support both chemical and biological synthesis.

### BACKGROUND OF THE INVENTION

Split-and-pool libraries of small molecules and biopolymers can be prepared via solid-phase chemistry and used in drug discovery campaigns. Each bead of these libraries is a spatially isolated, clonal population of a single library member that can be individually interrogated. Initial work with these libraries was limited to affinity-based interactions with the bead-tethered compounds. More recent work (Cochrane et al. ACS Comb. Sci. 2019, 21, 5, 425-435, Price et al. Anal. Chem. 2016, 88, 5, 2904-2911, Cochrane et al. Anal. Chem. 2017, 89, 24, 13227-13234, Hackler et al. Anal. Chem. 2016, 88, 5, 2904-2911) has established that these particles can be used to deliver compounds for both affinity and functional assays. While affinity driven selections can be conducted in bulk reactions, functional screening requires compartmentalization of individual particles in volumes small enough to result in high local concentrations, e.g., in microfluidic droplets.

Although microfluidics is capable of generating highly mono-disperse droplets and particles, these systems present a high barrier to entry as the fabrication, components and operational skills are highly specialized. Bulk emulsification, on the contrary, is available to every lab with minimal equipment and no specialized training require. However, bulk emulsification (e.g., via vortexing or shaking) generally results in droplet sizes that range over several orders of magnitude. Particle-templated emulsion (Hatori et al. Anal. Chem. 2018, 90, 16, 9813-9820) introduces mono-sized particles to the emulsion volume that serve as supports for droplet generation, yielding shells of the aqueous mixture around the particle support. This results in mono-disperse droplets surrounding the solid supports. But the final emulsion still contains a population of “satellite droplets” without particles.

Therefore, there is a need for particles that facilitate chemical reactions during library synthesis and that can be used in library screening. The method of production must scale to large numbers of beads (ideally on the scale of library synthesis or greater) while still yielding uniform particles.

### BRIEF SUMMARY

Provided herein is a composition comprising a particle comprising a magnetic core coated in a polymer gel. In some embodiments, the polymer gel comprises polyacrylamide. In some embodiments, the polymer gel comprises about 4% w/v to about 10% w/v polyacrylamide. In some embodiments, the polymer gel comprises between about 10:1 to about 40:1 acrylamide to bis acrylamide. In some embodiments, the polymer gel comprises 19:1 or 37.5:1 acrylamide to bis acrylamide.

In some embodiments, the particle further comprises an additive that inhibits phase separation, condensate formation, and/or coacervation. In some embodiments, the additive comprises an albumin. In some embodiments, the albumin is modified with one or more reactive groups.

In some embodiments, about 0.001 mM to about 20 mM of the polymer gel is functionalized. In some embodiments, the particle is fluorescently labeled. In some embodiments, the polymer gel has a pore size of about 20 nm to about 200 nm. In some embodiments, the magnetic core is a magnetic bead. In some embodiments, the magnetic bead has a diameter of about 0.5 μm to about 10 μm. In some embodiments, the magnetic bead has a diameter of about 1.0 μm, 2.8 μm, or 10.0 μm. In some embodiments, the total particle diameter is less than about 40 μm, optionally when the total particle diameter is about 6 μm to about 12 μm. In some embodiments, the composition comprises particles that have a uniform distribution of diameters. In some embodiments, the composition comprises particles with a particle diameter coefficient of variance of about 3% to about 50%. In some embodiments, the average total diameter of the particles is about 5 μm to about 10 μm. In some embodiments, the average total diameter of the particles is about 7 μm. In some embodiments, at least 95% of the total particles in the composition comprise a magnetic core.

Provided herein, in some embodiments, is a method of producing a composition comprising a particle comprising a magnetic core encapsulated in a polymer gel, comprising emulsifying an aqueous solution comprising monomers and magnetic beads with a solution comprising a polymerization initiator to cause polymerization of the monomer and thereby producing a composition comprising a magnetic core encapsulated in the polymer gel. In some embodiments, the polymer gel comprises polyacrylamide. In some embodiments, the method comprising emulsifying a solution comprising 4% w/v to 10% acrylamide monomer and magnetic beads with the solution comprising an initiator. In some embodiments, the solution comprising the monomer comprises acrylamide monomer and bis-acrylamide monomer. In some embodiments, the solution comprising the monomer and magnetic beads is an aqueous solution. In some embodiments, the initiator is in an oil solution, optionally wherein the initiator is TEMED. In some embodiments, the aqueous solution comprises ammonium persulfate. In some embodiments, emulsifying the solution comprises, vortexing, homogenizing, mixing, stirring, and/or shaking. In some embodiments, the initiator causes polymerization of the acrylamide to coat the magnetic beads in the polyacrylamide gel.

In some embodiments, the method further comprising combining the solution comprising acrylamide and bis-acrylamide monomer and magnetic beads and the solution comprising a polymerization initiator to produce a combined composition prior to emulsification. In some embodiments, the combined composition comprises an oil phase and an aqueous phase. In some embodiments, the method further comprising sparging the combined composition with inert gas. In some embodiments, the inert gas is argon.

In some embodiments, the method further comprising applying a magnetic field to the composition to separate the particles. In some embodiments, the method further comprising removing a supernatant that does not comprise the particles. In some embodiments, the method further comprising washing the particles. In some embodiments, the method further comprising resuspending the particles. In some embodiments, the method further comprising functionalizing a portion of the gel.

Provided herein is a particle produced by a method provided herein.

Provided herein is a library of particles produced by a method provided herein.

Provided herein, in some embodiments, is a kit comprising a composition comprising aqueous solution comprising magnetic beads and acrylamide and a composition comprising an oil solution comprising a polymerization initiator. The instructions for use of the kit are provided according to a method provided herein.

Provided herein is a kit comprising a particle provided herein and the instructions for use are provided according to a method provided herein.

### BRIEF DESCRIPTION OF THE DRAWINGS

FIGS. 1A-1E show a variety of gel particle functionalization by copolymerization. FIG. 1A shows the bank gel beads. Gel particles were copolymerized with 5′-methacrylamido oligonucleotide (FIG. 1B), methacrylamide-modified hairpin headpiece DNA (HDNA) (FIG. 1C), propargyl methacrylate (PMA) (FIG. 1D), or primary amines using 3-(aminopropyl) methacrylamide (APMA) (FIG. 1E). Gel functionalization was detected via chemical reactions with fluorescent dye-labeled complementary functional groups, including complementary oligonucleotide hybridization (FIG. 1B), enzymatic ligation of double-stranded (dsDNA) modules used for DEL synthesis (FIG. 1C), Cu(I)-catalyzed azide-alkyne cycloaddition (CuAAC) with Alexa Fluor 488 azide (488AF-N3) (FIG. 1D), or amine acylation with fluorescein succinimidyl ester (FAM-OSu) (FIG. 1E). Gel labeling was detected via flow cytometry and compared to blank gel beads (FIG. 1A-1E, right panel).

FIG. 2 shows the structure and characterization of methacrylamide-modified HDNA (ac-HDNA) structure and characterization. Product formation was confirmed by MALDI-TOF MS analysis of HPLC fractions. MS labels indicate the theoretical exact mass (top) and observed mass (bottom) for [M+H]⁺.

FIGS. 3A-3B show gel particles that were prepared with varying concentrations of APMA, acylated with FAM-OSu, analyzed by flow cytometry (FIG. 3A), and quantitated (gray boundary indicates standard error) (FIG. 3B).

FIG. 4A shows DNA-templated beads that were prepared by PCR using DNA oligonucleotide primer P1 (black dashed, single-sided arrow)-functionalized magnetic beads (black circle), where P1 sequence is set forth in SEQ ID NO:1 and DNA oligonucleotide primer P2 (black single-sided arrow), where P2 sequence is set forth in SEQ ID NO:3. DNA templates contained a T7 RNA polymerase promoter element (T7 prom, black arrow). Untemplated negative control magnetic beads were labeled with Alexa Fluor 647 (647AF, black hollow circle with black dots). Each bead set was encapsulated in P2-copolymerized hydrogel. FIG. 4B shows in vitro transcription of the gel-encapsulated DNA-templated and untemplated beads in the presence of FAM-labeled DNA oligonucleotide probe of the RNA transcript 5′ region (P3, black single-sided arrow), where P3 sequence is set forth in SEQ ID NO: 5. The P3 probe detected the presence of RNA transcript (black) hybridized via P2 in the gel. FIG. 4C shows two-dimensional flow cytometry analysis indicating that the majority of particles exhibited either exclusively red fluorescence (660 nm, untemplated negative controls in Q1) or green fluorescence (520 nm, templated and RNA-loaded beads in Q3).

FIG. 5A shows DNA-templated beads that were encapsulated in polyacrylamide hydrogel copolymerized with oligonucleotide primer P1 (black dashed, single-sided arrow). Transcribed mRNAs hybridize to gel-linked P1 and a 3′ puromycin-modified DNA oligonucleotide P4 (black “puro”), where P4 sequence is set forth in SEQ ID NO:6. The mRNA (black strand) encoded a ribosome binding site (RBS), epitope tag (gray), glycine-serine linker (G4S, gray) (SEQ ID NO:19), HiBiT luciferase complementation tag (black), and stop codon (*). Ribosomal protein synthesis terminated with incorporation of the 3′ puromycin into the nascent chain, tethering the translated peptide to P4. Epitopes were detected via immunofluorescence and HiBiT was quantitated via luciferase complementation with LgBiT. FIGS. 5B-5C show in-gel translation of FLAG-templated particles, which were probed using 647AF-labeled anti-FLAG antibody, visualized, and compared with FIG. 5D-5E untranslated particles in confocal imaging of antibody fluorescence (Texthem=650/720 nm) and magnetic bead autofluorescence (Texthem=490/560 nm). FIGS. 5B and 5D have a scale of 50 μm. FIGS. 5C and 5E show twice magnification of—FIGS. 5B and 5D, respectively. FIG. 5F shows IVTT reactions that were programmed with FLAG-, HA- or V5-templated particles, where FLAG encoding sequence is set forth in SEQ ID NO: 13, HA encoding sequence is set forth in SEQ ID NO: 12, and V5 encoding sequence is set forth in SEQ ID NO: 11, and translated epitopes detected with 647AF-anti-FLAG, APC-anti-HA or CF488A-anti-V5 immunofluorescence by flow cytometry (FLAG and HA: λₑₓ/λₑₘ=640/660 nm; V5: λ^{ex}/λₑₘ=488/530 nm; translated particles in black, untranslated particles in gray). FIG. 5G shows in-gel capture of translated FLAG, HA, and V5 epitopes with and without 10 μM P4 (black, gray) which was quantitated via HiBiT complementation. Error bars reflect standard deviation of the mean.

FIG. 6 shows sorting of hydrogel magnetic particles using fluorescence activated cell sorting (FACS). HA and V5 templated particles were spiked (1% each) into a background of library particles (NNK₅), where NNK₅ library template sequence is set forth in SEQ ID NO:16. The particles were then translated in a bulk IVT reaction, incubated with APC-labeled anti-HA and CF488A-labeled anti-V5 antibodies before FACS. After sorting, each population was PCR-amplified and sequenced by Next-Generation Sequencing (NGS).

FIG. 7 shows flow cytometry analysis of different diameter magnetic bead templates for gel particle formation. Magnetic beads were encapsulated in gel copolymerized with ac-P1 (20 μM), where ac-P1 sequence is set forth in SEQ ID NO:2. Populations 1-, 2.8-, and 10-μm-diameter magnetic beads encapsulated in hydrogel were gated via forward and side scatter correlation (top) and the gated population fluorescence intensities (bottom, λₑₓ/λₑₘ=490/530 nm) of unprobed (gray) and FAM-P1′-probed (black) samples were compared to confirm encapsulation.

FIG. 8 shows HiBiT quantitation assay calibration curve. Various concentrations of HiBiT peptide (0.1-1000 nM) were assayed for chemiluminescence upon addition of excess LgBiT complementation reagent.

FIG. 9 shows hydrogel particle diameters as a function of templating magnetic bead diameter. Box and whisker plots indicate mean, standard deviation, upper and lower quartiles (box), and 1.5× quartile range (whiskers).

FIG. 10A shows hydrogel particle size uniformity analyzed by confocal fluorescence microscopy. Scale=100 μm. FIG. 10B shows a magnified portion of FIG. 10A. FIG. 10C shows the diameters of hydrogel particles measured for 32k particles (median=7.3 μm). FIG. 10D shows flow cytometry (λex/λem=488/520 nm) used to analyze probed (black) and unprobed (gray) gel particles.

FIG. 11 shows the schematic illustration of the particle synthesis approach. (i) Magnetic beads are suspended in a monomer premix, a layer of initiator containing oil is added, and the suspension is emulsified. (ii) Droplets are cured to form hydrogel droplets. (iii) Bead-containing gel particles are isolated magnetically.

FIG. 12 shows the enrichment rates for the various control tags by sequencing the sorted hit particles. Reads were pattern matched to epitope tag sequences or the NNK₅ library degenerate sequence. The HA positive hit pool was ˜99% HA-encoding sequences and the V5 hit pool was ˜50% V5-encoding sequence, a ˜100-fold and ˜50-fold enrichment from the library starting material.

FIG. 13 shows hydrogels prepared by bulk emulsification scale with the size of the templating magnetic bead (e.g., 1.0 μm, 2.8 μm, or 10.0 μm). Particle dispersity is inversely proportional to the magnetic bead diameter.

FIG. 14 shows translation and detection of non-canonical amino acid-containing gel particle library beads. NNU library particles (3×10⁶) were subjected to an engineered IVT reaction that was recoded to install an azido-lysine (AzK) at CUG codons. Translated particles were washed and treated with an AF647-alkyne in a CuAAC reaction before analysis by flow cytometry. Particles were sorted to isolate the top 2% of the AF647 population and the sequences compared to the starting library. A 6-fold enrichment was observed after one round of screening.

FIG. 15 shows NNU library particles (3×10⁶) prepared by emPCR subjecting to an engineered IVT reaction that was recoded to install an azido-lysine (AzK) at CUG codons. Translated particles were washed and incubated with an AF647-alkyne in a CuAAC reaction before analysis by flow cytometry. Particles were sorted by gating the top 2% of the AF647 signal.

FIG. 16A and FIG. 16B show qPCR analysis of emPCR library preparation. Aliquots of 100 beads were quantitated for to obtain average loading (dotted traces) and limiting dilution of beads was sampled in 77 wells (gray traces) to obtain single-bead quantitation of DNA templating (FIG. 16A). Traces for standards (light gray) and negative template control (black) are also shown in FIG. 16A. Quantitation of 100-bead and DNA-templated single beads is shown in the box plots (FIG. 16B). Average particle loading for the 100-bead aliquot was 4,200 DNA molecules per bead (FIG. 16B). Average particle loading for single DNA-templated beads was 42,000 DNA molecules per bead (FIG. 16B).

FIG. 17A shows the time-dependent quantitation of hydrogel particles loaded with the fluorogenic (turn-on) green fluorescent probe of trypsin activity. FIG. 17B shows the time-dependent quantitation of hydrogel particles loaded with an N-terminally-labeled tryptic peptide, wherein the N-terminal label is a red-fluorescent dye, Cy5. Upon tryptic digestion, Cy5 is liberated from the gel, resulting in a decrease in fluorescence signal over time (turn-off).

FIG. 18 shows schematic illustration of various copolymerized functionalities, including but not limited to crosslinkers, cell adhesion promoters (e.g., alkyl amines), affinity capture tags (e.g., chloroalkane HaloTag), enzyme capture, probe capture, and oligonucleotides for hybridization. The latter two are useful for labeling and characterization.

FIG. 19 shows gel particles copolymerized with oligonucleotide and subsequently hybridized with a trypsin activity-based probe conjugated to the complementary oligonucleotide, schematically illustrating that the probe-hybridized gel particles digested with trypsin, dequenching the activity-based probe and resulting in fluorescent gel particles.

### DETAILED DESCRIPTION OF THE INVENTION

In some embodiments, provided herein is a versatile hydrogel-based particle that supports both chemical and biological synthesis. Particle preparation via the bulk emulsification methods disclosed herein is straightforward, scales well, and yields relatively uniform size distribution. The hydrogel layer of the particle can display an array of functionalities that is commonly used in both conventional split-and-pool combinatorial chemical synthesis or templated enzymatic biosynthesis. (Gartner et al. Science, 2004, 305, 5690, 1601-1605; Gartner et al. J. Am. Chem. Soc. 2001, 123, 28, 6961-6963; Halpin et al. DNA Display I. Sequence-Encoded Routing of DNA Populations, PLOS Biol, 2004, 2 (7), e173; Halpin et al. DNA Display II. Genetic Manipulation of Combinatorial Chemistry Libraries for Small-Molecule Evolution, PLOS Biol, 2004, 2 (7), e174; Halpin et al. DNA Display III. Solid-Phase Organic Synthesis on Unprotected DNA, PLOS Biol, 2004, 2 (7), e175.) Furthermore, templated library preparation using this particle system via in vitro translation can be particularly advantageous because RNA transcripts and subsequently translated peptides are localized to their host particle, generating monoclonal beads for screening without further emulsification or other partitioning.

In some embodiments, the particle provided herein can be used for library screening using the particle system in conjugation with fluorescence-activated cell sorting (FACS) instrumentation. This new particle format introduces important handling advantages that can enable encoded library synthesis automation while also unlocking functional screening capabilities by way of polyvalent encoded library member display. (MacConnell et al. ACS Comb. Sci. 2017, 19, 3, 181-192; Komnatnyy et al. Chem. Commun., 2018, 54, 6759-6771.)

The present inventors have found that magnetic bead templated emulsion polymerization yielded surprisingly uniform particles that do not require microfluidics for either preparation or analysis, and the particles exhibit superb handling and biocompatibility.

“Building block” as used herein refers to a chemical building block that is attached to other chemical building blocks or may be attached to other such blocks. Building blocks are diverse chemical structures that feature one or more functional groups that are used variously for coupling via chemical synthesis and potentially interacting with the target of interest.

“Functional site” or “functional group” as used herein refers to a chemical group that patriciates in a reaction and can produce a linkage between two moieties. Examples of functional groups include, but are not limited to, —NH₂, —SH, —OH, —CO₂H, halide, —N₃, —CONH₂, etc.

“Particle” as used herein refers to a discrete complex comprising a magnetic core coated by a polymer.

“A” or “an” means one and more than one. For example, “a particle” include one, two, three, or more particles.

By “library” is meant a collection of molecules or chemical entities.

By “oligonucleotide” is meant a polymer of nucleotides having a 5′-terminus, a 3′-terminus, and one or more nucleotides at the internal position between the 5′- and 3′-termini. The oligonucleotide may include DNA, RNA, or any derivative thereof known in the art.

By “tag” or “oligonucleotide tag” is meant an oligonucleotide portion of the library at least part of which contains information to identify the particle and/or library. For example, an oligonucleotide tag may contain information that allows identification of an associated functional group. In some embodiments an oligonucleotide tag is used as a barcode.

### I. Particles

In some embodiments, provided herein are particles comprising a magnetic core coated in a polymer gel. In some embodiments, the particle comprises a hydrogel coating the magnetic core. In some embodiments, hydrogel refers to a substance formed when an organic polymer (natural or synthetic) is cross-linked via covalent, ionic, or hydrogen bonds to create a three-dimensional open-lattice structure that entraps water molecules to form a gel. In some embodiments, the hydrogel may be a biocompatible hydrogel which refers to a polymer that forms a gel that is not toxic to living cells and allows sufficient diffusion of oxygen and nutrients to entrapped cells to maintain viability.

In some embodiments, provided herein are particles comprising acrylamide droplets around magnetic particles. In some embodiments, the particle is prepared in a bulk emulsification to yield uniform hydrogel compartments that are readily polymerized and magnetically isolated. Acrylamide offers a straightforward polymerization mechanism already used in many chemistry and molecular biology labs and the polyacrylamide product is generally inert to chemical and biochemical reactions. This radical-mediated polymerization readily incorporates a variety of functionalities to the polymer matrix simply by including molecules with both the desired functionality and a vinyl group for polymerization. In some embodiments, the present inventors have prepared hydrogels with both chemical (e.g., amine, azide) and biochemical (oligonucleotide) groups and then demonstrated that these groups can participate in various reactions in the gel matrix (infra). Thus, in some embodiments, the particle comprises a polyacrylamide gel coating a magnetic core.

In some embodiments, provided herein is a particle comprising a magnetic core coated with a hydrogel layer. In some embodiments, a hydrogel layer comprising polyacrylamide can be easily synthesized, and it can prevent aggregation and inhibit adherence to tube walls. Polyacrylamide is also inert, and thus compatible with a range of different chemical and biochemical reactivities. Gel particles comprise polyacrylamide can be variously functionalized by adding different reagents to the acrylamide monomer solution such as propargyl methacrylate for “alkyne” functionality (which can be coupled with molecules containing the “azide” functionality), N-(3-aminopropyl) methacrylamide for “amine” functionality, methacrylamide (ac)-modified oligonucleotide P1 for “reverse primer” functionality, or ac-headpiece DNA for HDNA functionality (which is used in DNA-encoded library synthesis). In some embodiments, the hydrogel polymer layer comprises polyacrylamide, acrylamide and/or bis-acrylamide. In some embodiments, the hydrogel polymer layer includes polyacrylamide and/or acrylamide. In some embodiments, the hydrogel polymer layer includes polyacrylamide.

In some embodiments, the hydrogel or polymer layer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof.

In some embodiments, gel particles comprise aforementioned polymers or hydrogels around the magnetic beads can be variously functionalized by adding different reagents to the respective monomer solution such as by including molecules with both the desired functionality and a vinyl group for polymerization. In some embodiments, hydrogel layers around the magnetic bead with both chemical (e.g., alkyne, amine, azide) and biochemical (oligonucleotide) functional groups can be made. In some embodiments, the functionalized hydrogel or polymer layer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof.

In some embodiments, hydrogel particles with a magnetic core are prepared via emulsification of hydrogel monomer solution and magnetic microbeads. In some embodiments, polyacrylamide hydrogel particles with a magnetic core can be prepared by suspending magnetic beads in a monomer aqueous solution comprises acrylamide and bis-acrylamide and initiators such as TEMED may be added to the oil phase. Subsequently, the aqueous phase and oil phase may be mixed together to form emulsified suspension. In some embodiments, the magnetic beads are carboxylic acid-functionalized magnetic beads (M-270 carboxylic acid Dynabeads, 5× 10⁷, ThermoFisher Scientific). In some embodiments, the ratio of acrylamide:bis-acrylamide in acrylamide monomer solution is 19:1 or 37.5:1 or any ratio between about 10:1 to about 40:1. In some embodiments, the oil/aqueous sample may be emulsified using a bead mill homogenizer (65 s, 2500 rpm, BeadBug, Benchmark Scientific, Sayreville, NJ), vortexing, or stirring. In some embodiments, the emulsified suspension may be polymerized on ice, and the hydrogel particles with magnetic cores can be isolated on a magnet after the polymerization.

In some embodiments, the ratio of acrylamide:bis-acrylamide in acrylamide monomer solution used for the preparation of hydrogel particles with magnetic cores is about 10:1, 10.5:1, 11:1, 11.5:1, 12:1, 12.5:1, 13:1, 13.5:1, 14:1, 14.5:1, 15:1, 15.5:1, 16:1, 16.5:1, 17:1, 17.5:1, 18:1, 18.5:1, 19:1, 19.5:1, 20:1, 20.5:1, 21:1, 21.5:1, 22:1, 22.5:1, 23:1, 23.5:1, 24:1, 24.5:1, 25:1, 25.5:1, 26:1, 26.5:1, 27:1, 27.5:1, 28:1, 28.5:1, 29:1, 29.5:1, 30:1, 30.5:1, 31:1, 31.5:1, 32:1, 32.5:1, 33:1, 33.5:1, 34:1, 34.5:1, 35:1, 35.5:1, 36:1, 36.5:1, 37:1, 37.5:1, 38:1, 38.5:1, 39:1, 39.5:1, or 40:1, or a ratio within a range defined by any two of the aforementioned ratios.

In some embodiments, the particle and/or the composition comprising the particle comprises an additive to inhibit phase separation, condensate formation, and/or coacervation of the hydrogel layer of the hydrogel particles with magnetic cores. In some embodiments, the additive comprises an albumin, such as bovine serum albumin (BSA) and acrylamide-modified BSA. In another embodiments, albumin is modified with one or more reactive groups such as albumin modified with vinyl groups and acrylamide-modified BSA. In some embodiments, the particle comprises bovine serum albumin (BSA), modified BSA, human serum albumin (HSA), and/or modified HSA.

In some embodiments, the hydrogel polymer layer comprises about 70-96% weight/volume (w/v) fluid, such as water, and about 4-30% w/v polymer. Unless stated otherwise, the percentage is weight/volume. In some embodiments, the hydrogel layer of the hydrogel particle with a magnetic core comprises about 70% fluid and 30% polymer, 72% fluid and 28% polymer, 74% fluid and 26% polymer, 76% fluid and 24% polymer, 78% fluid and 22% polymer, 80% fluid and 20% polymer, 82% fluid and 18% polymer, 84% fluid and 16% polymer, 86% fluid and 14% polymer, 88% fluid and 12% polymer, 90% fluid and 10% polymer, 92% fluid and 8% polymer, 94% fluid and 6% polymer, or 96% fluid and 4% polymer, or a percentage of fluid or polymer within a range defined by any two of the aforementioned percentages. In some embodiments, the polymer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof.

In certain embodiments, the fluid in the hydrogel polymer layer of the hydrogel particle with a magnetic core comprises water, or any other solvents that are compatible with the hydrogel. In some embodiments, the water content of the hydrogel layer is about 90%, 91%, 92%, 93%, 94%, 95%, or 96% or a percentage within a range defined by any two of the aforementioned percentages. In some embodiments, the hydrogel polymer layer of the hydrogel particle with a magnetic core comprises about 10% polymer, 9% polymer, 8% polymer, 7% polymer, 6% polymer, 5% polymer, or 4% polymer, or a percentage within a range defined by any two of the aforementioned percentages. In some embodiments, the hydrogel polymer layer of the hydrogel particle with a magnetic core comprises about 10% polyacrylamide, 9% polyacrylamide, 8% polyacrylamide, 7% polyacrylamide, 6% polyacrylamide, 5% polyacrylamide, or 4% polyacrylamide, or a percentage within a range defined by any two of the aforementioned percentages.

In some embodiments, the particle comprises pores in the polymer that allow for diffusion in an out of the gel. In some embodiments, the pore is the pore size is selected such that a reagent, substrate, reactant, and/or enzyme is retained in the polymer. In some embodiments, the pore size is selected such that a high local concentration of reactants, substrates, or enzymes is produced in the hydrogel to facilitate proximity driven synthesis. In some embodiments, the pore size is about 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, or 200 nm or a size within a range defined by any two of the aforementioned sizes. In some embodiments, the hydrogel polymers form a hydrogel matrix having pores (for example, a porous hydrogel matrix). These pores are capable of retaining sufficiently large genetic materials or peptides within the hydrogel matrix coated on the magnetic bead, but allow small materials, such as reagents, to pass through the pores, thereby passing in and out of the hydrogel matrix. In some embodiments, the pore size is determined by the ratio of the concentration of polymer to the concentration of crosslinker. In some embodiments, the ratio of polymer to crosslinker is 30:1, 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, or 1:30, or a ratio within a range defined by any two of the aforementioned ratios.

In some embodiments, magnetic core of the hydrogel comprises a magnetic bead. In some embodiments, the magnetic core is responsive to an external magnetic field but can be demagnetize when the field is removed. Thus, the paramagnetic microparticles can be efficiently separated from a solution using a magnet, but can be easily resuspended without magnetically induced aggregation occurring. In some embodiments, magnetic beads comprise a magnetite rich core (such as iron oxides) encapsulated by a pure polymer shell. In one embodiment, suitable magnetic beads comprise about 20-35% magnetite/encapsulation ratio. For example, magnetic beads comprising a magnetite/encapsulation ratio of about 23%, 25%, 28%, 30%, 32%, or 34% are suitable for use in the present invention.

In some embodiments, the magnetic bead inside the hydrogel particle has a diameter of about 0.5 μm to about 10 μm, such as, for example, 0.5, 1, 2, 3, 4, 5, 6, 7, 8, 9, or 10 μm, in diameter, or a diameter within a range defined by any two of the aforementioned values. In some embodiments, the magnetic bead inside the hydrogel particle has a diameter of about 2.8 μm.

In some embodiments, the magnetic beads used here can have a functional group coated surface. Methods for coating magnetic beads with functional groups, either directly or indirectly, are known in the art. For example, the functional groups (e.g., COOH) can coat a magnetic bead during formation of the magnetic beads. See, for example, U.S. Pat. No. 5,648,124. In additional, magnetic beads can be coated with functional groups by covalently coupling a functional group (one or more) to a COOH group (one or more) on the magnetic bead. A particular example of a functional group coated surface is a surface which is coated with moieties which each have a free functional group which is bound to the amino group of the amino silane of the magnetic bead; as a result, the surfaces of the magnetic beads are coated with the functional group containing moieties. In particular, carboxylic acid-coated magnetic beads are commercially available. Other functional groups can be coated on the magnetic beads, such as, but not limited to, amino-coated, carboxyl-coated and encapsulated carboxyl group-coated magnetic beads. In some embodiments, other functional groups can be coupled to the magnetic beads through carbodiimide coupling to carboxy groups on the surface of the magnetic beads. Other functional groups include, but not limited to, an amine group, a carboxyl group, an encapsulated carboxyl group, silica (SiOH), and diethyl aminoethyl (DEAE).

In some embodiments, the particle is about 5 μm to about 10 μm in diameter. In some embodiments, the particle is a sufficient size to enable multiple synthetic reactions to take place in the polymer coating. In some embodiments, the overall diameters of the hydrogel particles are measured by using confocal fluorescence microscopy. In some embodiments, the median diameter is 7±2 μm. The overall hydrogel particle's diameter is less than about 40 μm. In some embodiments, the total particle diameter is less than about 40 μm, optionally when the total particle diameter is about 6 μm to about 12 μm. In some embodiments, the overall diameter of the hydrogel particles with magnetic cores is about 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32μ, 33 μm, 34μ, 35 μm, 36 μm, 37 μm, 38 μm, 39 μm, or a diameter within a range defined by any two of the aforementioned values, for example about 2 μm to about 30 μm, about 3 μm to about 20 μm, about 4 μm to about 10 μm, or about 5 μm to about 9 μm.

In some embodiments, hydrogel particles with magnetic cores have a wide variety of functionalities embedded in the hydrogel layer or hydrogel matrix. In some embodiments, gel particles can be variously functionalized by adding the following reagents to the acrylamide monomer solution including propargyl methacrylate (PMA) or propargyl methacrylate analog or other alkyne-derivatized or -containing compounds known to the skilled artisan for the “alkyne” functionality. N-(3-aminopropyl) methacrylamide (APMA) or N-(3-aminopropyl) methacrylamide analog or other amine derivatized or containing compounds known to the skilled artisan for the “amine” functionality, 5′-methacrylamide-modified DNA oligonucleotide P1(acrydite) or acrydite-P1(ac-P1) or acrydite-P1 analog or other acrydite derivatized compounds known to the skilled artisan for the “reverse primer” functionality, or methacrylamide DNA Headpiece or ac-hairpin headpiece DNA (HDNA) or its analog for the “HDNA” functionality, or aminoethyldithio- or sulfhydryl-derivatized compounds or its analog known to the skilled artisan for the “sulfhydryl” functionality, or any combination thereof.

In some embodiments, hydrogel particles with magnetic cores have a wide variety of functionalities embedded in the hydrogel layer or hydrogel matrix. In some embodiments, gel particles can be variously functionalized by adding the following reagents to the specific gel's monomer solution including propargyl methacrylate or propargyl methacrylate analog or other alkyne derivatized or containing compounds known to the skilled artisan for the “alkyne” functionality. N-(3-aminopropyl) methacrylamide or N-(3-aminopropyl) methacrylamide analog or other amine derivatized or containing compounds known to the skilled artisan for the “amine” functionality, 5′-methacrylamide-modified DNA oligonucleotide P1(acrydite) or acrydite-P1 or acrydite-P1 analog or other acrydite derivatized compounds known to the skilled artisan for the “reverse primer” functionality, or methacrylamide DNA Headpiece or ac-hairpin headpiece DNA (HDNA) or its analog for the “HDNA” functionality, or aminoethyldithio- or sulfhydryl-derivatized compounds or analog known to the skilled artisan for the “sulfhydryl” functionality, or any combination thereof.

In some embodiments, the hydrogel or polymer layer comprises one or more reagents comprising propargyl methacrylate (PMA) or propargyl methacrylate analog or other alkyne derivatized or containing compounds known to the skilled artisan, N-(3-aminopropyl) methacrylamide (APMA) or N-(3-aminopropyl) methacrylamide analog or other amine derivatized or containing compounds known to the skilled artisan, 5′-methacrylamide-modified DNA oligonucleotide P1(acrydite) or acrydite-P1 or acrydite-P1 analog or other acrydite derivatized compounds known to the skilled artisan, or methacrylamide DNA Headpiece or ac-hairpin headpiece DNA (HDNA) or its analog, or aminoethyldithio- or sulfhydryl-derivatized compounds or its analog known to the skilled artisan. In some embodiments, the hydrogel or polymer layer of the hydrogel particle is labeled with different fluorescent labels, such as appropriate fluorescent dye-labeled substrates, comprising complementary oligonucleotide, double-stranded oligonucleotide ligation module, azide, or succinimidyl ester.

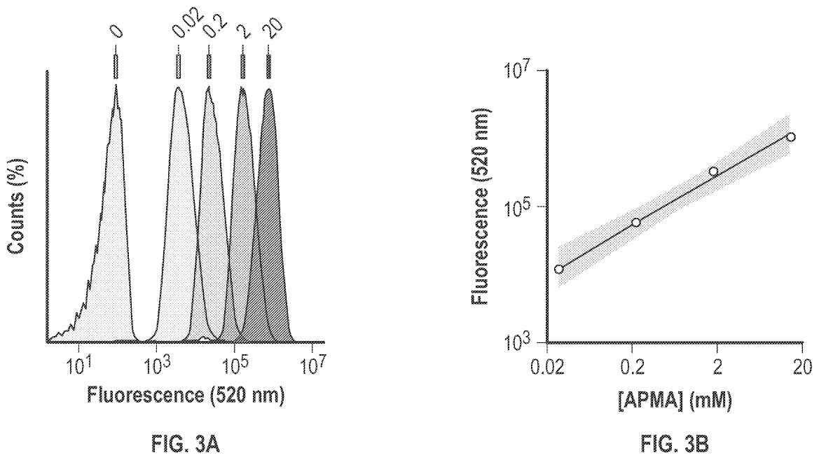

In some embodiments, hydrogel particle functionalization can be detected by flow cytometry with the addition of the appropriate fluorescent dye-labeled substrates, comprising complementary oligonucleotide, double-stranded oligonucleotide ligation module, azide, or succinimidyl ester. In some embodiments, hybridization and amine acylation result in the largest shift but all functionalization reactions result in baseline separation of product and starting material by flow cytometry. In some embodiments, hydrogels with escalating amine loading capacity can be prepared by copolymerizing increasing concentrations of APMA. In some embodiments, the APMA concentration is about 0.02 mM, 0.04 mM, 0.06 mM, 0.08 mM, 0.10 mM, 0.12 mM, 0.14 mM, 0.16 mM, 0.18 mM, 0.20 mM, 0.22 mM, 0.24 mM, 0.26 mM, 0.28 mM, 0.30 mM, 0.32 mM, 0.34 mM, 0.36 mM, 0.38 mM, 0.40 mM, 0.42 mM, 0.44 mM, 0.46 mM, 0.48 mM, 0.50 mM, 0.55 mM, 0.60 mM, 0.65 mM, 0.70 mM, 0.75 mM, 0.80 mM, 0.85 mM, 0.90 mM, 0.95 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, 8.0 mM, 8.1 mM, 8.2 mM, 8.3 mM, 8.4 mM, 8.5 mM, 8.6 mM, 8.7 mM, 8.8 mM, 8.9 mM, 9.0 mM, 9.1 mM, 9.2 mM, 9.3 mM, 9.4 mM, 9.5 mM, 9.6 mM, 9.7 mM, 9.8 mM, 9.9 mM, 10.0 mM, 10.5 mM, 11.0 mM, 11.5 mM, 12.0 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, or a concentration within a range defined by any two of the aforementioned values. In some embodiments, gel particles can be prepared with varying APMA concentrations, acylated with FAM-OSu, analyzed by flow cytometry, and quantitated as shown in FIG. 3A and FIG. 3B. In some embodiments, acylation with FAM-OSu resulted in log-linear increases in gel particle fluorescence over a 4 order of magnitude range.

In some embodiments, hydrogel particles with magnetic cores have a wide variety of functionalities embedded in the hydrogel layer or hydrogel matrix. In some embodiments, gel particles can be variously functionalized by adding the following reagents to the specific gel's monomer solution including propargyl methacrylate or propargyl methacrylate analog or other alkyne derivatized or containing compounds known to the skilled artisan for the “alkyne” functionality. N-(3-aminopropyl) methacrylamide or N-(3-aminopropyl) methacrylamide analog or other amine derivatized or containing compounds known to the skilled artisan for the “amine” functionality, 5′-methacrylamide-modified (acrydite) DNA oligonucleotide P1 (ac-P1) or acrydite-P1 analog or other acrydite derivatized compounds known to the skilled artisan for the “reverse primer” functionality, or methacrylamide DNA Headpiece or ac-hairpin headpiece DNA (HDNA) or its analog for the “HDNA” functionality, or aminoethyldithio- or sulfhydryl-derivatized compounds or analog known to the skilled artisan for the “sulfhydryl” functionality, or any combination thereof. In some embodiments, about 0.001 mM to about 20 mM of the hydrogel or polymer gel is functionalized. In some embodiments, about 0.01 mM to about 20 mM of the hydrogel or polymer gel is functionalized. In some embodiments, about 0.1 mM to about 20 mM of the hydrogel or polymer gel is functionalized. In some embodiments, about 1 mM to about 20 mM of the hydrogel or polymer gel is functionalized. In some embodiments, about 10 mM to about 20 mM of the hydrogel or polymer gel is functionalized.

In some embodiments, the hydrogel or polymer layer of the hydrogel particle comprises a protein, an enzyme, a polynucleotide, oligonucleotide, a polysaccharide, a fluorophore, a lipid, or a supramolecular assembly.

In some embodiments, the concentration of the functionalized hydrogel or polymer is about 0.001 mM, 0.005 mM, 0.01 mM, 0.015 mM, 0.02 mM, 0.025 mM, 0.03 mM, 0.035 mM, 0.04 mM, 0.045 mM, 0.05 mM, 0.055 mM, 0.06 mM, 0.065 mM, 0.07 mM, 0.075 mM, 0.08 mM, 0.085 mM, 0.09 mM, 0.095 mM, 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM, 18 mM, 19 mM, 20 mM, or a concentration within a range defined by any two of the aforementioned values.

In some embodiments, hydrogels can be diversely functionalized by including sub-stoichiometric methacrylamide-modified additives in the acrylamide:bis-acrylamide monomer solution for copolymerization. For example, incorporation of the synthetic hairpin headpiece DNA (HDNA) and subsequent enzymatic oligonucleotide ligation reaction demonstrate the viability of standard DEL synthesis workflows using these particles. The commercially available NH₂-HDNA can be readily transformed to the methacrylamide analog for copolymerization. Amine functionality can be routinely employed in solid-phase synthesis and also DEL synthesis. In some embodiments, particle loading capacity is quantitative over 4 orders of magnitude. For example, at the highest site density of (i.e., 20 mM) of APMA functionalized hydrogel particles, each median particle (about 7 μm diameter) particle harbors about 4 fmol amine sites.

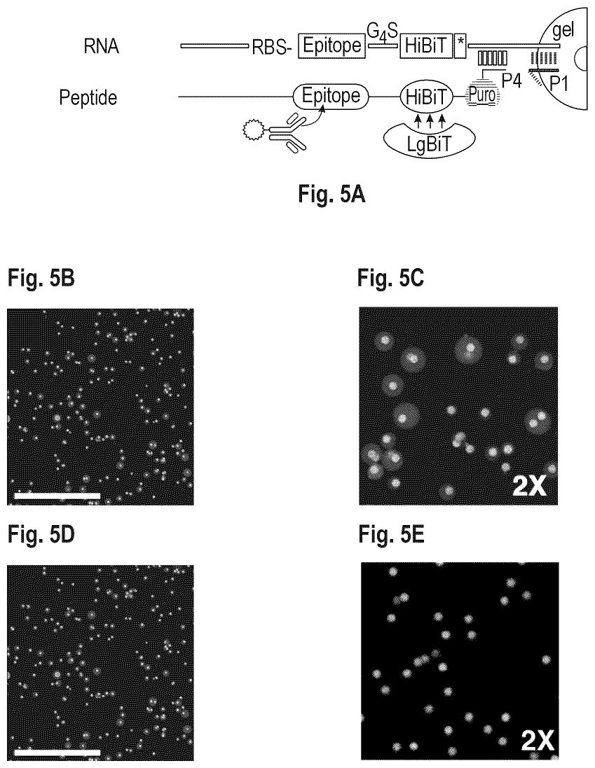

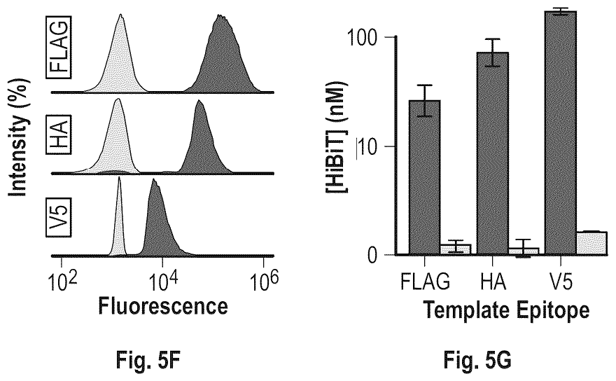

In some embodiments, beads can be templated with DNA encoding various affinity tag epitopes, such as FLAG, HA, or V5, fused to a HiBiT luciferase complementation tag and encapsulated in P1-functionalized gels. The gel particles are subjected to mRNA display-type in vitro transcription/translation reactions incorporating a puromycin-modified peptide capture oligonucleotide P4 complementary to the RNA directly 3′ of the stop codon (FIG. 5A). Translated beads can be analyzed by imaging microscopy and flow cytometry to visualize specific epitope translation. Translated gel particles exhibits homogeneous antibody-derived fluorescence throughout the gel periphery (FIGS. 5B-5C); only magnetic bead autofluorescence is detected in the same particles before translation (FIG. 5D). Translated gel particle fluorescence via flow cytometry is baseline-separated compared to untranslated particles for all three example epitopes (FIG. 5F). In some embodiments, in-gel peptide capture yield can be quantitated via HiBiT luminescence. In some embodiments, particles translated in the presence of puromycin capture oligonucleotide P4 retained about 100 nM HiBiT peptide, while smaller than 1 nM is retained in translations lacking P4 (FIG. 5G).

In some embodiments, gel particles comprise aforementioned polymers or hydrogels around the magnetic beads can be variously functionalized by adding different reagents to the respective monomer solution such as by including molecules with both the desired functionality and a vinyl group for polymerization. In some embodiments, hydrogel layers around the magnetic bead with both chemical (e.g., alkyne, amine, azide) and biochemical (oligonucleotide) functional groups can be made. In some embodiments, the functionalized hydrogel or polymer layer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. In some embodiments, the hydrogel particle's diameter coefficient of variance is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a percentage defined by any two of the aforementioned values.

In some embodiments, gel particles comprise aforementioned polymers or hydrogels around the magnetic beads can be variously functionalized by adding different reagents to the respective monomer solution such as by including molecules with both the desired functionality and a vinyl group for polymerization. In some embodiments, hydrogel layers around the magnetic bead with both chemical (e.g., alkyne, amine, azide) and biochemical (oligonucleotide) functional groups can be made. In some embodiments, the functionalized hydrogel or polymer layer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. In some embodiments, the average total diameter of the hydrogel particles is about 5.1 μm, 5.2 μm. 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm. 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, or a diameter defined by any two of the aforementioned values. In some embodiments, the average diameter of the hydrogel particles is about 7 μm.

In some embodiments, gel particles comprise aforementioned polymers or hydrogels around the magnetic beads can be variously functionalized by adding different reagents to the respective monomer solution such as by including molecules with both the desired functionality and a vinyl group for polymerization. In some embodiments, hydrogel layers around the magnetic bead with both chemical (e.g., alkyne, amine, azide) and biochemical (oligonucleotide) functional groups can be made. In some embodiments, the functionalized hydrogel or polymer layer comprises polyacrylamide, polyethylene glycol (PEG)-thiol, PEG-acrylate, acrylamide, N,N′-bis(acryloyl) cystamine (BACy), PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bis-acrylamide, diacrylate, diallylamine, triallylamine, divinyl sulfone, diethyleneglycol diallyl ether, ethylene glycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. In some embodiments, at least 95% of the total hydrogel particles in the composition comprise a magnetic core. In some embodiments, the percentage of the total hydrogel particles in the composition comprising a magnetic core is 95%, 95.5%, 96%, 96.5%, 97%, 97.5%, 98%, 98.5%, 99%, 99.5%, 100%, or a percentage defined by any two of the aforementioned values.

In some embodiments, a library of the hydrogel particles with magnetic cores comprises about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10₁₁, 7×10₁₁, 8×10₁₁, 9×10₁₁, 1×10₁₂ particles, or a number defined by any two of the aforementioned values.

In some embodiments, a library of the hydrogel particles with magnetic cores comprises about 1×10⁶ to 1×10¹² particles, 1×10⁶ to 1×10¹¹ particles, 1×10⁶ to 1×10¹⁰ particles, 1×10⁶ to 1×10⁹ particles, 1×10⁶ to 1×10⁸ particles, 1×10⁶ to 1×10⁷ particles, 1×10⁷ to 1×10¹² particles, 1×10⁷ to 1×10¹¹ particles, 1×10⁷ to 1×10¹⁰ particles, 1×10⁷ to 1×10⁸ particles, 1×10⁸ to 1×10¹² particles, 1×10⁸ to 1×10¹¹ particles, 1×10⁸ to 1×10¹⁰ particles, 1×10⁸ to 1×10⁹ particles, 1×10⁹ to 1×10¹² particles, 1×10⁹ to 1×10¹¹ particles, 1×10⁹ to 1×10¹⁰ particles, 1×10¹⁰ to 1×10¹² particles, or 1×10¹¹ to 1×10¹² particles.

### II. Methods of Making Particles

In some embodiment, provided herein is a method of producing a composition comprising a particle comprising a magnetic core encapsulated in a polymer gel. In some embodiments, the methods provided herein are advantageous in that they produce gel encapsulated nanoparticles in a single step by emulsifying an aqueous solution and an oil solution and do not require complicated assembly techniques or machinery. In some embodiments, the methods provided herein are amenable to producing gel encapsulated nanoparticles in a high throughput method and/or producing the gel encapsulated nanoparticles in bulk.

In some embodiments, the methods provided herein comprise emulsifying an aqueous solution comprising monomers and magnetic beads with a solution comprising a polymerization initiator to cause polymerization of the monomer. In some embodiments, the monomer is a monomer unit of a polymer. In some embodiments, the aqueous solution comprises acrylamide monomer. In some embodiments, the aqueous solution comprises about 4% to about 10%, about 5% to about 9%, about 6% to about 8%, or about 7% to about 10% acrylamide monomer.

In some embodiments, the aqueous solution comprises bis acrylamide. In some embodiments, the aqueous solution comprises a mixture of bis acrylamide in and acrylamide monomer. In some embodiments, the ratio of bis acrylamide to acrylamide is between about 10:1 to about 40:1, such as about 15:1 to about 20:1, about 30:1 to about 40:1, about 18:1 to about 38:1, or about 35:1 to about 40:1. In some embodiments, the ratio of bis acrylamide to acrylamide is about 17:1, about 18:1, about 19:1, about 20:1 or about 21:1. In some embodiments, the ratio of bis acrylamide to acrylamide is about 36:1, about 37:1, about 37.5:1, about 38:1, about 39:1 or about 40:1.

In some embodiments, the aqueous solution further comprises a second polymerization initiator that initiates polymerization when mixed with the solution comprising the polymerization inhibitor. In embodiments, the initiator is ammonium persulfate (APS) or TEMED. In some embodiments, the aqueous solution comprises APS and the oil solution comprises TEMED. In some embodiments, the aqueous solution comprises TEMED and the oil solution comprises TEMED. In some embodiments, APS and TEMED cause polymerization of the acrylamide and bisacrylamide to encapsulate the magnetic beads. In other embodiments, initiation of polymerization could be achieved using alternative radical sources (e.g., azobisisobutyronitrile, AIBN) or stimulus-responsive species (e.g., light-sensitive photoinitiators, such as dimethoxyphenylacetophenone, DMPA).

In some embodiments, the method comprises combining two solutions, such as an aqueous solution and an oil solution. In some embodiments, the aqueous solution comprises acrylamide and/or bis acrylamide and the oil solution comprises a polymerization initiator. In some embodiments, the two solutions are combined by mixing, emulsification, vortexing, homogenizing, stirring and/or shaking. In some embodiments, the method comprises combining the solutions using a mechanical system. In some embodiments, the method comprises combining the solutions using a vortexer, a rotating mixer, or a stir bar. In some embodiments, the method comprises manually shaking a container comprising the two solutions.

In some embodiments, the solutions are mixed over a period of time. In some embodiments, the solutions are mixed for a sufficient time to combine the solutions. In some embodiments, the solutions are mixed for a sufficient amount of time to allow polymerization of the monomer and encapsulation of the beads. In some embodiments the solutions are combined, mixed, vortexed, emulsified, homonogized, stirred, and/or shaken for at least one, at least two, at least three, at least four, at least 5, at least 10 minutes, at least 20 minutes, at least 30 minutes, at least 45 minutes, or at least 60 minutes. In some embodiments, the solutions are combined, mixed, vortexed, emulsified, homonogized, stirred, and/or shaken for one to 60 minutes, one to 45 minutes, one to 30 minutes, one to 20 minutes, one to 10 minutes, or one to five minutes. In some embodiments, the solutions are combined, mixed, vortexed, emulsified, homonogized, stirred, and/or shaken for 10 to 60 minutes, 20 to 60 minutes, 30 to 60 minutes, or 45 to 60 minutes.

In some embodiments, the solutions are mixed at room temperate. In some embodiments, the solutions are mixed at above room temperature. In some embodiments, the solutions are mixed at below room temperature. In some embodiments, the solutions are mixed at about 15° C. to about 25° C., about 17° C. to about 23° C., or about 20° C. to about 22° C.

In some embodiments, the method further comprises sparging the combined composition with gas. In some embodiments, the combined composition is sparged with an inert gas. In some embodiments, the combined composition is sparged with nitrogen, argon or helium. In some embodiments, the combined composition is sparged with inert gas for at least at least two, at least three, at least four, at least 5, at least 10 minutes, at least 20 minutes, at least 30 minutes, at least 45 minutes, or at least 60 minutes. In some embodiments, the combined composition is sparged for one to 60 minutes, one to 45 minutes, one to 30 minutes, one to 20 minutes, one to 10 minutes, or one to five minutes. In some embodiments, the combined composition is sparged for 10 to 60 minutes, 20 to 60 minutes, 30 to 60 minutes, or 45 to 60 minutes.

In some embodiments, the method further comprises applying a magnetic field to the combined composition to purify or isolate particles that comprise one or more magnetic beads encapsulated in a hydrogel. In some embodiments, applying a magnetic field to the combined mixture causes localization of the beads using the magnet while solution is removed or exchanged. In some embodiments, application of a magnetic field is used to separate particles that comprises one or more magnetic beads encapsulated in a hydrogel from particles that do not comprise one or more magnetic beads. In some embodiments, particles that have two or more, three or more, or four or more magnetic beads are separated from particles or gel that does not comprise a magnetic bead. In some embodiments, application of a magnetic field allows isolation of a population of homogenous particles that contain one or more different reagents. In some embodiments, application of a magnetic field produces a library of particles that each comprise a different reagent.

In some embodiments, after a magnetic field is applied to a combined composition, particles that comprise a magnetic bead are removed from solution. In some embodiments, particles that comprise a magnetic bead form a pellet and/or are localized to a portion of a tube or container containing the combined solution. In some embodiments, the magnetic field causes precipitation of particles comprising a magnetic bead. In some embodiments, a supernatant that does not comprise particles comprising a magnetic bead is aspirated and/or removed from the combined composition. In some embodiments, aspiration of the supernatant results in purification of particles comprising a magnetic bead.

In some embodiments, following application of the magnetic field, particles comprising one or more magnetic beads are substantially purified from particles that do not comprises a magnetic bead. In some embodiments, following application of the magnetic field, the ratio of particles that comprise a magnetic bead to those that do not is at least 100:1, at least 1,000:1, or at least 10,000:1. In some embodiments, following application of the magnetic field, particles comprising a single magnetic bead are separated from particles that comprise multiple magnetic beads.

In some embodiments, following aspiration of the supernatant, the remaining particles that comprise one or more magnetic beads are washed one or more times. In some embodiments, the washes remove particles that do not comprise a magnetic bead. In some embodiments, the wash solution comprises a neutral buffed solution. In some embodiments, an excess of buffered solution is used to resuspend the particles, a magnetic field is applied, and the supernatant is aspirated, one or more time, resulting in washing of the particles. In some embodiments, following washing the ratio of particles that comprise a magnetic bead to those that do not is at least 100:1, at least 1,000:1, or at least 10,000:1. In some embodiments, the method comprises washing the beads, one, two, three, four, five times or more.

In some embodiments, following application of the magnetic field the particles are resuspended. In some embodiments, the particles are resuspended in a buffered solution. In some embodiments, the particles are resuspended in a buffered solution suitable for a chemical or biochemical reaction.

In some embodiments, the method comprises producing a functionalized gel, or a functionalized portion of the gel. In some embodiments, the gel is functionalized after particles comprising a polymer gel coating a magnetic core are purified, for examples, by magnetic separation. In some embodiments, the gel is functionalized prior to purification of encapsulated particles. In some embodiments, a portion of monomers are functionalized prior to polymerization. In some embodiments, the polymerized gel is functionalized.

In some embodiments, a functionalized gel or particle is one that comprises a functional group that is the substrate for a further reaction or synthetic method. For example, suitable functional groups comprise an oligonucleotide, a synthetic hairpin headpiece DNA, an alkyne, and/or a primary amine.

In some embodiments, particles are functionalized by copolymerization. In some embodiments, particles are copolymerized with a 5′ methacrylamide oligonucleotide, methacrylamide-modified HDNA, propargyl methacrylate, or APMA. In some embodiments, the gel is functionalized. In some embodiments, the gel is functionalized prior to polymerization. In some embodiments, the gel is functionalized at the same time as polymerization. In some embodiments, the gel is functionalized after polymerization.

In some embodiments, the magnetic bead is functionalized. In some embodiments, the magnetic bead is functionalized a 5′ methacrylamide oligonucleotide, methacrylamide-modified HDNA, propargyl methacrylate, or APMA. In some embodiments, the magnetic bead is functionalized prior to polymerization of the gel encapsulating the bead. In some embodiments, the magnetic bead is functionalized at the same that the gel is polymerized. In some embodiments, the magnetic bead is functionalized after the gel is polymerized. In some embodiments, the magnetic bead is carboxylic acid functionalized.

In some embodiments, the gel is functionalized by adding compounds comprising a functional group to a monomer solution. In some embodiments, the reagents are added under conditions that allow coupling of the functional group to the gel. In some embodiments, the compounds are incubated with the monomer solution such that at least a portion of the monomer is functionalized. In some embodiments, at least 0.01%, at least 0.1%, at least 1% or at least 10% of the monomers are functionalized.

In some embodiments, the particles or monomers are functionalized using an excess of functional agent. In some embodiments, the particles are functionalized by incubation with about 0.0001 mM to about 100 mM (such as about 0.0002 mM to about 50 mM, about 0.0002 mM to about 20 mM, about 1 mM to about 100 mM, 5 mM to about 80 mM, about 10 mM to about 50 mM, about 10 mM to about 40 mM, or about 15 mM to about 30 mM) of a compound comprising a functional group. In some embodiments, the particles are functionalized by incubation with about 20 mM of a compound comprising a functional group. In some embodiments, the compound comprising a functional group is propargyl methacrylate.

In some embodiments, the gel and/or bead is functionalized such that a particle comprises one or more functional groups. In some embodiments, a given particle contains multiple copies of the same functional group. In some embodiments, a given particle contains multiple copies of different functional groups. In some embodiments, different particles in a mixture of particles comprise different functional groups. In some embodiments, each particle in a mixture comprises the same functional group.

In some embodiments, functionalization of the gel results in particles that comprise a template for a synthesis reaction. For example in some embodiments, functionalization of the gel results in particles that comprise a HDDNA or oligonucleotide template.

In some embodiments, prior to polymerization, the solution comprising a monomer and magnetic beads comprises one or more reagents. In some embodiments, the one or more reagents comprises a protein, an enzyme, a polynucleotide, oligonucleotide, a polysaccharide, a fluorophore, or a lipid. In some embodiments, the reagent is an enzyme capable of synthesis. In some embodiments, the reagent comprises a polynucleotide or oligonucleotide substrate for a synthetic reaction. In some embodiments, the reagent comprises a fluorophore that can be used to detect the particle. In some embodiments, the solution comprises multiple reagents, for example a fluorophore and an oligonucleotide.

In some embodiments, polymerization of the monomer causes the one or more reagents to be incorporated in the gel. In some embodiments, the reagents are covalently linked to the bead and/or the gel. In some embodiments, the reagents are encapsulated in the gel. In some embodiments, the reagents are non-covalently held within the gel.

In some embodiments, the method comprises polymerizing a monomer to produce a particle comprises magnetic bead coated with a gel and subsequently functionalizing the formed particles. In some embodiments, the particles are functionalized using an excess of functional agent. In some embodiments, the particles are functionalized by incubation with about 0.0001 mM to about 100 mM (such as about 0.0002 mM to about 50 mM, about 0.0002 mM to about 20 mM, about 1 mM to about 100 mM, 5 mM to about 80 mM, about 10 mM to about 50 mM, about 10 mM to about 40 mM, or about 15 mM to about 30 mM) of a compound comprising a functional group. In some embodiments, the particles are functionalized by incubation with about 20 mM of a compound comprising a functional group. In some embodiments, the compound comprising a functional group is propargyl methacrylate.

In some embodiments, the method further comprises detecting functionalization of the gel and/or the bead. In some embodiments, detection is using a chemical reaction with florescent dye-labeled functional groups. In some embodiments, the detection is using complementary oligonucleotide hybridization, enzymatic ligation of dsDNA molecules, CuAAC with Alexa Fluor 488 azide (488A-FN3), or amine acylation with fluorescein succinimidyl ester (FAM-OSu).

In some embodiments, the methods provided herein result in a library of particles. In some embodiments, the library of particles comprises a plurality of particles with different functional groups. In some embodiments, the library of particles comprises a plurality of particles with the same functional groups.

In some embodiments, the methods provided herein are compatible with different magnetic bead sizes such as about 1.0, 2.8, or 10.0 μm in diameter. In some embodiments, the methods provided herein result in a gel particle with a magnetic core size about 1, 2.8, or 10 μm in diameter after encapsulation.

In some embodiments, increasing the magnetic bead size forms larger and more uniform gel particle distributions using the methods provided herein. In some embodiments, for hydrogels prepared by bulk emulsification scale with different templating magnetic bead sizes, such as about 1.0, 2.8, or 10.0 μm in diameter, the resulting overall hydrogel particle size dispersity is inversely proportional to the magnetic bead diameter. For example, in some embodiments, when the templating magnetic bead size is about 1 μm in diameter, the mean diameter of the hydrogel particles after encapsulation of the magnetic bead is about 4.9 μm in diameter with a standard deviation of about 1.8 μm and a coefficient of variation of about 36% by counting 447 particles. In some other embodiments, when the templating magnetic bead size is about 2.8 μm in diameter, the mean diameter of the hydrogel particles after encapsulation of the magnetic bead is about 7.5 μm in diameter with a standard deviation of about 1.9 μm and a coefficient of variation of 25% by counting 390 particles. In some other embodiments, when the templating magnetic bead size is about 10 μm in diameter, the mean diameter of the hydrogel particles after encapsulation of the magnetic bead is about 12 μm in diameter with a standard deviation of about 2.0 μm and a coefficient of variation of 16% by counting 66 particles.

In some embodiments, the hydrogel particle with a 1 μm in diameter magnetic core prepared by the methods provided herein has a hydrogel volume of about 88 fL. In some embodiments, the hydrogel particle with a 2.8 μm in diameter magnetic core prepared by the methods provided herein has a hydrogel volume of about 260 fL. In some embodiments, the hydrogel particle with a 10 μm in diameter magnetic core prepared by the methods provided herein has a hydrogel volume of about 570 fL.

### III. Methods of Detecting a Synthetic Product

Also provided herein are methods of detecting a synthetic product of a reaction in a particle. In some embodiments, advantageously, the particles and methods provided herein result in concentration of reactants within a particle and retention of a synthetic product. In some embodiments, the method comprises synthesizing a synthetic product in a particle. In some embodiments, the particle comprises a gel coating a magnetic core. In some embodiments, the particle comprises a synthetic template. In some embodiments, the gel is a polymeric gel.

In some embodiments, the method comprises detecting a synthetic product of a reaction in a particle comprising a polyacrylamide gel coating a magnetic bead. In some embodiments, the polymeric gel facilitates proximity-driven synthesis by the enzyme. In some embodiments, the polymeric gel increases the local concentration of the substrate, enzyme, and template, resulting in production of the synthetic product. In some embodiments, one or more of the enzyme, substrate, and template are attached to the polymer gel or the bead. In some embodiments, one or more of the enzyme, substrate, and template are trapped within the polymeric gel. In some embodiments, the pore size of the polymeric gel is such that the enzyme, substrate, and/or template are retained.

In some embodiments, the method comprises providing a synthetic enzyme and a synthetic substrate within the polymeric gel. In some embodiments, the synthetic substrate is a nucleic acid or an amino acid. In some embodiments, the synthetic substrate is a nucleotide. In some embodiments, the synthetic substrate is a tRNA. In some embodiments, the synthetic substrate is a naturally-occurring tRNA or a synthetic tRNA. In some embodiments, the synthetic substrate is a tRNA for any of the naturally occurring amino acids.

In some embodiments, the enzyme incorporates the substrate into a synthetic product. In some embodiments, the substrate is incorporated into a polymeric product. In some embodiments, the substrate is incorporated into a nucleic acid or protein product. In some embodiments, the sequence of the synthetic product is determined by the sequence of the template. For example, in some embodiments, the template is an mRNA and the synthetic product is a protein comprising the corresponding amino acid sequence. In some embodiments, the template is a DNA or RNA sequence and the synthetic product is a complementary DNA or RNA sequence.

In some embodiments, a functionalized particle is further modified to produce a particle comprising a template. In some embodiments, a templated particle is produced. In some embodiments, the particle is functionalized with a primer. In some embodiments, the primer is incubated with template DNA and oligonucleotides under PCR conditions to produce a templated particle comprising double stranded DNA. In some embodiments, the particle is templated following polymerization of the gel and purification of the gel-encapsulated particles. In some embodiments, the magnetic bead is functionalized and templated. In some embodiments, the polymeric gel is functionalized and templated. In some embodiments, the monomer if functionalized before or after polymerization.

In some embodiments, the number or percentage of templated particles is determined. In some embodiments, templated particles are detected by qPCR using a primer that hybridizes to the DNA synthesized during templating. In some embodiments, templated particles are detected by binding of a fluorescent probe to the template. In some embodiments, templated particles are detected by binding of an antibody to the template. In some embodiments, templated particles are purified from non-templated particles.

In some embodiments, the template is a single stranded DNA. In some embodiments, the template is a double stranded DNA. In some embodiments, the template comprises a transcription initiation sequence. In some embodiments, the template comprises a transcription termination sequence. In some embodiments, the template comprises a promoter sequence for an RNA polymerase. In some embodiments, the template comprises a promoter sequence for T7 RNA polymerase.

In some embodiments, the template is an mRNA. In some embodiments, the template contains a translation imitation sequence, such as AUG. In some embodiments, the template comprises a ribosome binding site. In some embodiments, the template comprises a transcription termination sequence. In some embodiments, the template comprise a puromycin translation termination sequence. In some embodiments, the template comprises a HiBit tag. In some embodiments, the template comprises a sequence encoding an epitope tag. In some embodiments, the template comprises a sequence encoding an amino acid tag, for example a 6HIS or FLAG tag (SEQ ID NO:21). In some embodiments, the template comprises one or more tags or epitopes separated by a flexible linker.

In some embodiments, the substrate is a naturally occurring nucleotide. In some embodiments, the substrate is any of adenosine, thymidine, guanosine, cytidine, uridine, deoxyadenosine, deoxythymidine, deoxyguanosine, deoxycytidine, inosine, or diamino purine), base analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), modified bases (e.g., 2′-substituted nucleotides, such as 2′-O-methylated bases and 2′-fluoro bases), intercalated bases, modified sugars (e.g., 2′-fluororibose; ribose; 2′-deoxyribose; arabinose; hexose; anhydrohexitol; altritol; mannitol; cyclohexanyl; cyclohexenyl; morpholino that also has a phosphoramidate backbone; locked nucleic acids (LNA, e.g., where the 2′-hydroxyl of the ribose is connected by a Ci-β alkylene or Ci-6 heteroalkylene bridge to the 4′-carbon of the same ribose sugar, where exemplary bridges included methylene, propylene, ether, or amino bridges); glycol nucleic acid (GNA, e.g., R-GNA or S-GNA, where ribose is replaced by glycol units attached to phosphodiester bonds); threose nucleic acid (TNA, where ribose is replace with a-Lthreofuranosyl-(3′->2′)); and/or replacement of the oxygen in ribose (e.g., with S, Se, or alkylene, such as methylene or ethylene)), modified backbones (e.g., peptide nucleic acid (PNA), where 2-amino-ethyl-glycine linkages replace the ribose and phosphodiester backbone), and/or modified phosphate groups (e.g., phosphorothioates, 5′-N-phosphoramidites, phosphoroselenates, boranophosphates, boranophosphate esters, hydrogen phosphonates, phosphoramidates, phosphorodiamidates, alkyl or aryl phosphonates, phosphotriesters, bridged phosphoramidates, bridged phosphorothioates, and bridged methylene-phosphonates). The oligonucleotide can be single-stranded (e.g., hairpin), double-stranded, or possess other secondary or tertiary structures (e.g., stemloop structures, double helixes, triplexes, quadruplexes, etc.). In some embodiments, the particle comprises a mixture of oligonucleotide substrates (e.g. A, C, G, and T or U). In some embodiments, the particle comprises a mixture of oligonucleotide substrates at about equal amounts.

In some embodiments, the enzyme is DNA or RNA polymerase. In some embodiments, the enzyme is DNA polymerase I. T7 DNA polymerase, DNA polymerase II, DNA polymerase VI, Taq DNA polymerase, T4 DNA polymerase, RNA polymerase I, DNA polymerase III, RNA polymerase II, RNA polymerase III, T7 RNA polymerase, or reverse transcriptase. In some embodiments, the enzyme is a ribosome. In some embodiments, the enzyme is from a microorganism such as bacteria, virus, or yeast.

In some embodiments, the synthetic product is such as a peptide that is 50 amino acids or less in length. In some embodiments, the synthetic product is a polypeptide that is more than 50 amino acids in length. In some embodiments, the synthetic product comprises one or more amino acids linked by peptide bonds. In some embodiments the synthetic product is about 2 to about 1000 amino acids in length. In some embodiments, the synthetic product is about 2 to about 50, about 2 to about 40, about 2 to about 30, about 2 to about 20, or about 2 to about 10 amino acids in length. In some embodiments, the synthetic product is a naturally occurring or synthetic peptide.

In some embodiments, the synthetic product is an oligonucleotide, such as a polynucleotide. In some embodiments, the synthetic product is double stranded DNA. In some embodiments, the synthetic product is single stranded DNA or RNA. In some embodiments, the synthetic product comprises two or more nucleotides linked by a phosphodiester bond. In some embodiments, the synthetic product comprises two or more nucleotides linked by a synthetic backbone. In some embodiments, the synthetic product is an oligonucleotide of about 2 to about 100 nucleotides in length. In some embodiments, the synthetic product is about 2 to about 100, about 2 to about 80, about 2 to about 60, about 2 to about 40, or about 2 to about 20 nucleotides in length. In some embodiments, the synthetic product is a naturally occurring or synthetic oligonucleotide.

In some embodiments, the synthetic product is a drug candidate. In some embodiments, the synthetic product is a drug target. In some embodiments, the synthetic product is a portion of a larger molecule. For example, in some embodiments, the synthetic protein is a portion of a protein.

In some embodiments, the template is conjugated to the magnetic bead. In some embodiments, the template is covalently linked to the magnetic bead. In some embodiments, the template is conjugated to the magnetic bead prior to polymerization of the gel around the bead. In some embodiments, the templated is conjugated to the bead after polymerization. In some embodiments, the template is directly conjugated to the bead. In some embodiments, the template is conjugated to the bead by a linker. In some embodiments, the linker is a small molecule, peptide, or oligonucleotide linker.

In some embodiments, the template is conjugated to the polymer gel. In some embodiments, the template is covalently linked to the polymer gel. In some embodiments, the template is non-covalently retained within the polymer gel. In some embodiments, the template is conjugated to a monomer prior to polymerization of the gel around the bead. In some embodiments, the templated is conjugated to the polymer following polymerization. In some embodiments, the template is directly conjugated to the polymer. In some embodiments, the template is conjugated to the polymer by a linker. In some embodiments, the linker is a small molecule, peptide, or oligonucleotide linker.

In some embodiments, the synthetic product remains associated with the particle following synthesis. In some embodiments, the synthetic product does not diffuse into solution surrounding the particle. In some embodiments, the synthetic product is retained in the particle non-covalently. In some embodiments, the particle pore size of the polymer gel is such that the synthetic product cannot escape the polymer gel. In some embodiments, the synthetic product interacts non-covalently with the template or enzyme, resulting in its retention within the particle. In some embodiments, ribosomal stalling results in retention of the synthetic product. In some embodiments, the synthetic product comprises a 3′ puromycin resulting in tethering of the product to the template.

In some embodiments, the method further comprises detecting the activity of the enzyme. In some embodiments, the presence or amount of the synthetic product is detected. In some embodiments, a detection or selection assay is performed. In some embodiments, enzyme activity is measured using flow cytometry, a cell sorting device, measuring a density, detecting an affinity tag, or DNA sequencing. In some embodiments, the activity is measured by binding a fluorescently labeled protein, for example, an antibody. In some embodiments, the activity is measured by binding a fluorescently labeled oligonucleotide. In some embodiments, the labeled oligonucleotide is complementary to the synthetic product or a portion of the synthetic product. IN some embodiments, the labeled oligonucleotide is not complementary to the template.

In some embodiments, the method further comprises detecting the synthetic product. For example, in some embodiments, the method comprises detecting the synthetic product using a fluorescent label and performing flow cytometry or cell sorting to detect the product. In some embodiments, the synthetic product is a polynucleotide and the polynucleotide product is detected using sequencing.

### IV. Methods of Producing a DNA-Encoded Library

Also provided herein are methods of producing a DNA-encoded library (DEL). In some embodiments, the DEL comprises particles, wherein each particle comprises a different DNA sequence. In some embodiments, the DNA sequence is associated with a building block. In some embodiments, the DNA or oligonucleotide sequence associated with a building block is a “tag.” In some embodiments, the DNA sequence is associated with a chemical building block, such as functional group. In some embodiments, the DNA is used as a barcode or tag to identify a small molecule associated with the particle.

In some embodiments, the method comprises incubating a particle comprising a magnetic core coated in a polyacrylamide gel comprising a functional site and a synthetic hairpin headpiece DNA (HDNA) under conditions that couple a building block to the functional site. A previous publication provides an example of a DNA headpiece, where beads are functionalized with azido DNA headpiece moieties (MacConnell et al. ACS Comb. Sci. 2015, 17, 9, 518-534). In particular embodiments, the headpiece includes an oligonucleotide selected from the group consisting of a double-stranded oligonucleotide, a single-stranded oligonucleotide, or a hairpin oligonucleotide. In some embodiments, the headpiece includes a primer-binding region.

In some embodiments, the library includes a plurality of headpieces. In some embodiments, each headpiece of the plurality of headpieces includes an identical sequence region (e.g., a primer-binding region) and a different encoding region (e.g., a first tag that encodes for use of the library, identity of the library, a linkage, a spacer, or addition of a first component or an oligonucleotide sequence that facilitates hybridization, amplification, or sequencing technologies).

Generally, the headpiece includes a non-self-complementary sequence on the 5′- or 3′-terminus that allows for binding an oligonucleotide tag by polymerization, enzymatic ligation, or chemical reaction. The headpiece can allow for ligation of oligonucleotide tags and optional purification and phosphorylation steps. After the addition of the last tag, an additional adapter sequence can be added to the 5′-terminus of the last tag. Exemplary adapter sequences include a primer-binding sequence or a sequence having a label (e.g., biotin). In cases where many building blocks and corresponding tags are used (e.g., 100), a mix-and-split strategy may be employed during the oligonucleotide synthesis step to create the necessary number of tags. Such mix-and-split strategies for DNA synthesis are known in the art. The resultant library members can be amplified by PCR following selection for binding entities versus a target(s) of interest.

In some embodiments, the headpiece comprises one or more primer-binding sequences. For example, the headpiece comprises a sequence in the loop region of the hairpin that serves as a primer-binding region for amplification, where the primer-binding region has a higher melting temperature for its complementary primer (e.g., which can include flanking identifier regions) than for a sequence in the headpiece. In other embodiments, the complex includes two primer-binding sequences (e.g., to enable a PCR reaction) on either side of one or more tags that encode one or more building blocks. Alternatively, the headpiece may contain one primer-binding sequence on the 5′- or 3′-terminus. In some embodiments, the headpiece is a hairpin, and the loop region forms a primer-binding site or the primer-binding site is introduced through hybridization of an oligonucleotide to the headpiece on the 3′ side of the loop. A primer oligonucleotide, containing a region homologous to the 3′-terminus of the headpiece and carrying a primer-binding region on its 5′-terminus (e.g., to enable a PCR reaction) may be hybridized to the headpiece and may contain a tag that encodes a building block or the addition of a building block. The primer oligonucleotide may contain additional information, such as a region of randomized nucleotides, e.g., 2 to 16 nucleotides in length, which is included for bioinformatics analysis.

The headpiece can optionally include a hairpin structure, where this structure can be achieved by any useful method. For example, the headpiece can include complementary bases that form intermolecular base pairing partners, such as by Watson-Crick DNA base pairing and/or by wobble base pairing. In another example, the headpiece can include modified or substituted nucleotides that can form higher affinity duplex formations compared to unmodified nucleotides, such modified or substituted nucleotides being known in the art. In some embodiments, the headpiece includes one or more cross-linked bases to form the hairpin structure. In some embodiments, bases within a single strand or bases in different double strands can be cross-linked, e.g., by using psoralen.

In some embodiments, the headpiece or tag comprises one or more labels that allow for detection. For example, the headpiece, one or more oligonucleotide tags, and/or one or more primer sequences comprises an isotope, a radioimaging agent, a marker, a tracer, a fluorescent label (e.g., rhodamine or fluorescein), a chemiluminescent label, a quantum dot, and/or an affinity tag such as biotin or his tag.

In some embodiments, the headpiece or tag is modified to increase solubility in semi-, reduced-, or non-aqueous (e.g., organic) conditions. In some embodiments, the C5 positions of T or C bases are modified with aliphatic chains without significantly disrupting their ability to hydrogen bond to their complementary bases. Exemplary modified or substituted nucleotides are 5′dimethoxytrityl-N4-diisobutylaminomethylidene-5-(1-propynyl)-2′-deoxycytidine,3′-[(2-cyanoethyl)-(N,Ndiisopropyl)]-phosphoramidite; 5′-dimethoxytrityl-5-(1-propynyl)-2′-deoxyuridine.3′-[(2-cyanoethyl)-(N,Ndiisopropyl)]-phosphoramidite; 5′-dimethoxytrityl-5-fluoro-2′-deoxyuridine,3′-[(2-cyanoethyl)-(N,N-diisopropyl)]phosphoramidite; and 5′-dimethoxytrityl-5-(pyren-1-yl-ethynyl)-2′-deoxyuridine, or 3′-[(2-cyanoethyl)-(N,N15 diisopropyl)]-phosphoramidite.

In addition, the headpiece and/or oligonucleotide tag can be interspersed with modifications that promote solubility in organic solvents. For example, azobenzene phosphoramidite can introduce a hydrophobic moiety into the headpiece design. Such insertions of hydrophobic amidites into the headpiece can occur anywhere in the molecule. Thus, addition of hydrophobic residues into the headpiece design allows for improved solubility in semi- or non-aqueous (e.g., organic) conditions, while rendering the headpiece competent for oligonucleotide tagging. Furthermore, DNA tags that are subsequently introduced into the library can also be modified at the C5 position of T or C bases such that they also render the library more hydrophobic and soluble in organic solvents for subsequent steps of library synthesis.

Various ligation techniques can be used to add scaffolds, building blocks, spacers, linkages, tags, and/or the headpiece to produce a complex. Accordingly, any of the binding steps described herein can include any useful ligation techniques, such as enzymatic ligation and/or chemical ligation. These binding steps can include the addition of one or more tags to the headpiece or complex: the addition of a spacer to the headpiece; and the addition of one or more scaffolds or building blocks to the headpiece or complex. In particular embodiments, the ligation techniques used for any oligonucleotide provide a resultant product that can be transcribed and/or reverse transcribed to allow for decoding of the library or for template-dependent polymerization with one or more DNA or RNA polymerases.

Generally, enzymatic ligation produces an oligonucleotide having a native phosphodiester bond that can be transcribed and/or reverse transcribed. Exemplary methods of enzyme ligation are provided herein and include the use of one or more RNA or DNA ligases, such as T4 RNA ligase, T4 DNA ligase, CircLigase™ ssDNA ligase, CircLigase™ II ssDNA ligase, and ThermoPhage™ ssDNA ligase (Prokazyme Ltd., Reykjavik, Iceland). In some embodiments, ligation includes the use of an RNA ligase or a combination of an RNA ligase and a DNA ligase. Ligation can further include one or more soluble multivalent cations, in combination with one or more ligases.

Chemical ligation can also be used to produce oligonucleotides capable of being transcribed or reverse transcribed. The efficacy of a chemical ligation technique to provide oligonucleotides capable of being transcribed or reverse transcribed may need to be tested. This efficacy can be tested by any useful method, such as liquid chromatography-mass spectrometry, RT-PCR analysis, and/or PCR analysis. In particular embodiments, chemical 5 ligation includes the use of one or more chemical-reactive pairs to provide a spacing moiety that can be transcribed or reverse transcribed. In particular, reactions suitable for chemical-reactive pairs are preferred candidates for the ligation process (Kolb et al., Angew. Chem. Int. Ed., 40:2004-2021 (2001); Van der Eycken et al., QSAR Comb. Sci., 26:1115-1326 (2007)). In one embodiment, the ligated oligonucleotides contain a linkage that polymerases have a reduced ability to read or translocate through, e.g. an “unreadable” linkage.

In some embodiments, the methods described herein comprise reaction conditions that promote enzymatic or chemical ligation between the headpiece and a tag or between two tags. These reaction conditions include using modified nucleotides within the tag, as described herein; using donor tags and acceptor tags having different lengths and varying the concentration of the tags; using different types of ligases, as well as combinations thereof (e.g., CircLigase™ DNA ligase and/or T4 RNA ligase), and varying their concentration; using poly ethylene glycols (PEGs) having different molecular weights and varying their concentration; use of non-PEG crowding agents (e.g., betaine or bovine serum albumin); varying the temperature and duration for ligation; varying the concentration of various agents, including ATP, Co(NH3)6Ch, and yeast inorganic pyrophosphate; using enzymatically or chemically 20 phosphorylated oligonucleotide tags; using 3′-protected tags; and using preadenylated tags. These reaction conditions also include chemical ligations.

The headpiece and/or tags can include one or more modified or substituted nucleotides. In preferred embodiments, the headpiece and/or tags include one or more modified or substituted nucleotides that promote enzymatic ligation, such as 2′-O-methyl nucleotides (e.g., 2′-O-methyl guanine or 2′-O-methyl uracil), 2′-fluoro 25 nucleotides, or any other modified nucleotides that are utilized as a substrate for ligation. In some embodiments, the headpiece and/or tags are modified to include one or more chemically reactive groups to support chemical ligation (e.g. an optionally substituted alkynyl group and an optionally substituted azido group). In some embodiments, the tag oligonucleotides are functionalized at both termini with chemically reactive groups, and, optionally, one of these termini is protected, such that side-reactions may be reduced (e.g., reduced polymerization side-reactions).

In some embodiments, before or after ligation, the complex is be purified. In some embodiments, the complex is purified to remove unreacted headpiece or tags that may result in cross-reactions. In some embodiments, the complex is purified to remove any reagents or unreacted starting material that can inhibit or lower the ligation activity of a ligase. For example, the presence of phosphate may result in lowered ligation activity. In some embodiments, entities that are introduced into a chemical or ligation step may need to be removed to enable the subsequent chemical or ligation step.

Enzymatic and chemical ligation can include poly ethylene glycol having an average molecular weight of more than 300 Daltons (e.g., more than 600 Daltons, 3,000 Daltons, 4,000 Daltons, or 4,500 Daltons). In some embodiments, the poly ethylene glycol has an average molecular weight from about 3,000 Daltons to 9,000 Daltons. In some embodiments poly ethylene glycol can be present in any useful amount, such as from about 25% (w/v) to about 35% (w/v), such as 30% (w/v).

In some embodiments, the tags are installed by ligation of a single-stranded oligonucleotide to a single-stranded oligonucleotide.

Branched oligomeric or polymeric compounds can also be synthesized provided that at least one building block comprises three functional groups which are reactive with other building blocks. A library of the disclosure can comprise linear molecules, branched molecules or a combination thereof.

In some embodiments, a building block refers to a chemical building block that is attached to other chemical building blocks or may be attached to other such blocks. In some embodiments, a building block is a portion of an organic small molecule. When the functional moiety is polymeric or oligomeric, the structural unit is a monomeric unit of a multimer or oligomer. A structural unit may also include a skeletal structure (e.g., a skeletal structural unit) that is connected or may be connected to one or more additional structures (e.g., a peripheral structural unit). In some embodiments, the building blocks are complementary (i.e., the building blocks must be able to react together to form a structure comprising two or more building blocks). In some embodiments, the building blocks have at least two reactive groups. In some embodiments, the building block has only one reactive group. In some embodiments, reactive groups on two different building blocks are complementary, i.e. capable of reacting together to form a covalent bond. Example of building blocks are provided in U.S. patent application publication No. 2007/0224607, which is incorporated herein by reference.

In addition, chemical-reactive pairs (or functional groups) can be readily included in synthesis schemes and will support the efficient chemical ligation of oligonucleotides. In addition, the resultant ligated oligonucleotides can act as templates for template-dependent polymerization with one or more polymerases. In some embodiments, any of the binding steps described herein for tagging encoded libraries can be modified to include enzymatic ligation and/or chemical ligation techniques.

In some embodiments, the method comprises providing a building block and an associated oligonucleotide under conditions that allow for attachment of the building block to the particle and oligonucleotide to the headpiece DNA or to an oligonucleotide attached to the headpiece DNA. In some embodiments, the conditions are ligation conditions. In some embodiments, the method comprises incubation with DNA ligase. In some embodiments, the oligonucleotide is attached to the HDNA or an oligonucleotide attached to the HDNA by polymerization. In some embodiments, the oligonucleotide is attached by chemical reaction. In some embodiments, oligonucleotides are ligated by ligated use of chemical-reactive pairs (e.g., a pair including optionally substituted alkynyl and azido functional groups) and/or enzyme ligation, such as use of one of more RNA ligases and/or DNA ligases.

In another aspect, the invention features a library including one or more particles described herein. In some embodiments, the library includes a plurality of headpieces. In other embodiments, each headpiece of the plurality of headpieces comprises an identical sequence region (e.g., a primer-binding region) and a different encoding region (e.g., a tag that encodes for identity of the library). In particular embodiments, the library includes between about 10² to 10²⁰ particles (e.g., about 10² to 10³, 10² to 10⁴, 10² to 10⁵, 10² to 10⁶, 10² to 10⁷, 10² to 10⁸, 10² to 10⁹, 10² to 10¹⁰, 10² to 10¹¹, 10² to 10¹², 10² to 10¹³, 10² to 10¹⁴, 10² to 10¹⁵, 10² to 10¹⁶, 10² to 10¹⁷, 10² to 10¹⁸, 10² to 10¹⁹, 10⁴ to 10⁸, 10⁴ to 10⁶, 10⁴ to 10⁷, 10⁴ to 10⁸, 10⁴ to 10⁹, 10⁴ to 10¹⁰, 10⁴ to 10, 10⁴ to 10¹², 10⁴ to 10¹³, 10⁴ to 10¹⁴, 10⁴ to 10¹⁵, 10⁴ to 10¹⁶, 10⁴ to 10¹⁷, 10⁴ to 10¹⁸, 10⁴ to 10¹⁹, 10⁴ to 10²⁰, 10⁵ to 10⁶, 10⁵ to 10⁷, 10⁵ to 10⁸, 10⁵ to 35 10⁹, 10⁵ to 10¹⁰, 10⁵ to 10¹¹, 10⁵ to 10¹², 10⁵ to 10¹³, 10⁵ to 10¹⁴, 10⁵ to 10¹⁵, 10⁵ to 10¹⁶, 10⁵ to 10¹⁷, 10⁵ to 10¹⁸, 10⁵ to 10¹⁹, or 10⁵ to 10²⁰). In some embodiments, each particle is different.

In any of the above embodiments, the method further comprises one or more steps to diversify the library or to interrogate the members of the library, as described herein. In some embodiments, the method further comprises identifying a small drug-like library member that binds or inactivates a protein of therapeutic interest. In other embodiments, the method further comprises contacting a member of the library with a biological target under conditions suitable for at least one member of the library to bind to the target, removing one or more library members that do not bind to the target, and analyzing the one or more oligonucleotide tags associated with the target.

In any of the above embodiments, the encoded information is provided in one or more tags or in a combination of more than one tag. In some embodiments, the encoded information is represented by more than one tag (e.g., two, three, four, five, six, seven, eight, nine, ten, or more tags). In some embodiments, the encoded information is represented by more than one tag, where all encoding tags are contained within the encoding sequence (e.g., by using of a specific tag combination to encode information). In some embodiments, the encoded information is represented by more than one tag, where less than all encoding tags are contained within the encoding sequence (e.g., by using one tag from a set of more than one individual tag to encode within an individual encoding sequence).

In any of the above embodiments, the headpiece, and/or the oligonucleotide tag comprises from about 5 to about 300 nucleotides. In some embodiments, the headpiece, and/or the oligonucleotide tag comprises from 5 to 250 nucleotides, from 5 to 200 nucleotides, from 5 to 150 nucleotides, from 5 to 100 nucleotides, from 5 to 90 nucleotides, from 5 to 80 nucleotides, from 5 to 70 nucleotides, from 5 to 60 nucleotides, from 5 to 50 nucleotides, from 5 to 40 nucleotides, from 5 to 30 nucleotides, from 5 to 20 nucleotides, from 5 to 10 nucleotides, from 10 to 300 nucleotides, from 10 to 250 nucleotides, from 10 to 200 nucleotides, from 10 to 150 nucleotides, from 10 to 100 nucleotides, from 10 to 50 nucleotides, from 10 to 25 nucleotides, from 20 to 300 nucleotides, from 20 to 250 nucleotides, from 20 to 200 nucleotides, from 20 to 150 nucleotides, from 20 to 100 nucleotides, from 20 to 50 nucleotides, from 30 to 300 nucleotides, from 30 to 250 nucleotides, from 30 to 200 nucleotides, from 30 to 150 nucleotides, from 30 to 100 nucleotides, from 30 to 50 nucleotides, from 40 to 300 nucleotides, from 40 to 250 nucleotides, from 40 to 200 nucleotides, from 40 to 150 nucleotides, from 40 to 100 nucleotides, from 40 to 75 nucleotides, from 40 to 50 nucleotides, from 50 to 300 nucleotides, from 50 to 250 nucleotides, from 50 to 200 nucleotides, from 50 to 150 nucleotides, from 50 to 100 nucleotides, from 50 to 75 nucleotides, from 60 to 300 nucleotides, from 60 to 250 nucleotides, from 60 to 200 nucleotides, from 60 to 150 nucleotides, from 60 to 100 nucleotides, from 60 to 75 nucleotides, from 70 to 300 nucleotides, or from 70 to 250 nucleotides, from 70 to 200 nucleotides, from 70 to 150 nucleotides, from 70 to 100 nucleotides, from 80 to 300 nucleotides, from 80 to 250 nucleotides, from 80 to 200 nucleotides, from 80 to 150 nucleotides, from 80 to 100 nucleotides, from 90 to 300 nucleotides, from 90 to 250 nucleotides, from 90 to 200 nucleotides, from 90 to 150 nucleotides, or from 90 to 100 nucleotides, from 100 to 300 nucleotides, from 100 to 250 nucleotides, from 100 to 200 nucleotides, from 100 to 150 nucleotides, from 100 to 120 nucleotides, from 150 to 300 nucleotides, from 150 to 250 nucleotides, from 150 to 200 nucleotides, or from 200 to 300 nucleotides, from 200 to 250 nucleotides, and from 250 to 300 nucleotides.

In some embodiments, the HDNA is conjugated to the polymer gel. In some embodiments, the HDNA is conjugated to polyacrylamide. In some embodiments, the HDNA is conjugated to the magnetic core.

In some embodiments, the method comprises coupling the building block to a functional site located on or in the particle. In some embodiments, the functional site is located on the magnetic core. I some embodiments, the functional site is located in the polymer gel. In some embodiments, the functional site is reactive with one or more reactive groups of the building block. In some embodiments, the functional site comprises a boronate, amine, isocyanate, carboxylic acid, or aryl halide reactive group.

In some embodiments, the method of producing a DNA-encoded library comprises multiple rounds of incubations wherein each round comprises incubating a particle with a building block that attaches to a functional group in the particle and an oligonucleotide tag that attaches to the headpiece DNA or a previous oligonucleotide tag. In some embodiments, one or more clean up or purification steps is performed during or between each round. In some embodiments, the method results in a particle comprising multiple building blocks and a polynucleotide sequence comprising the associated tag for each building block. Accordingly, in some embodiments, the method comprises incubating the particle with a second oligonucleotide tag and second building block, a third oligonucleotide and third building block and so forth.

In some embodiments, the oligonucleotide sequence is associated with additional information such as the concentration of functional sites or different batches of particles.

In some embodiments, the method further comprises amplifying the oligonucleotide. In some embodiments, the oligonucleotide is amplified to product the library. In some embodiments, the oligonucleotide is sequenced to determine the building blocks present on a particle. In some embodiments, the sequencing determines additional information such as the concentration of functional sites or different batches of particles.

In yet another aspect, the invention features a method of screening a plurality of chemical entities, the method including: (a) contacting a target with any particle described herein and/or a library described herein; and (b) selecting one or more particles having a predetermined characteristic for the target, as compared to a control, thereby screening the chemical entity.

To create numerous chemical entities within the library, a solution containing the headpiece can be divided into multiple aliquots and then placed into a multiplicity of physically separate compartments, such as the wells of a multiwell plate. Generally, this is the “split” step. Within each compartment or well, successive chemical reaction and ligation steps are performed with a single-stranded tag within each aliquot. The relationship between the chemical reaction conditions and the sequence of the single-stranded tag are recorded. The reaction and ligation steps may be performed in any order. Then, the reacted and ligated aliquots are combined or “pooled,” and optionally purification may be performed at this point. These split and pool steps can be optionally repeated.

Next, the library can be tested and/or selected for a particular characteristic or function, as described herein. For example, the mixture of tagged chemical entities can be separated into at least two populations, where the first 10 population binds to a particular biological target and the second population does not (e.g., by negative selection or positive selection). The first population can then be selectively captured (e.g., by eluting on a column providing the target of interest or by incubating the aliquot with the target of interest) and, optionally, further analyzed or tested, such as with optional washing, purification, negative selection, positive selection, or separation steps.

In some embodiments, the library is contacted with a biological target under conditions suitable for at least one member of the library to bind to the target, followed by removal of library members that do not bind to the target, and analyzing the one or more oligonucleotide tags associated with the target. This method can optionally include amplifying the tags by methods known in the art. Exemplary biological targets include enzymes (e.g., kinases, phosphatases, methylases, demethylases, proteases, and DNA repair enzymes), proteins involved in protein-protein interactions (e.g., ligands for receptors), receptor targets (e.g., GPCRs and RTKs), ion channels, bacteria, viruses, parasites, DNA, RNA, prions, and carbohydrates.

In another embodiment, the chemical entities that bind to a target are not subjected to amplification but are analyzed directly. Exemplary methods of analysis include microarray analysis, including evanescent resonance photonic crystal analysis; bead-based methods for deconvoluting tags; label-free photonic crystal biosensor analysis; or hybridization-based approaches.

These methods can be used to identify and discover any number of chemical entities with a particular characteristic or function, e.g., in a selection step. The desired characteristic or function may be used as the basis for partitioning the library into at least two parts with the concomitant enrichment of at least one of the members or related members in the library with the desired function. In particular embodiments, the method comprises identifying a small drug-like library member that binds or inactivates a protein of therapeutic interest. In any of these instances, the oligonucleotide tags encode the chemical history of the library member and, in each case, a collection of chemical possibilities may be represented by any particular tag combination. In some embodiments, a sequence of chemical reactions is designed, and a set of building blocks is chosen so that the reaction of the chosen building blocks under the defined chemical conditions will generate a combinatorial plurality of molecules (or a library of molecules), where one or more molecules may have utility as a therapeutic agent for a particular disease.

### V. Methods of In Vitro Translation

Also provided herein are in vitro translation methods utilizing the particles provided herein. In some embodiments, the method comprises incubating a particle comprising a magnetic core coated with a polyacrylamide gel comprising an RNA template, ribosomes, and amino acids under conditions for translation of the RNA into a polypeptide.

In some embodiments, the RNA template is containing in or on the particle. In some embodiments, the RNA template is conjugated to the magnetic core. In some embodiments, the RNA template is conjugated to polyacrylamide gel. In some embodiments, transcribed RNA is captured by hybridization in the gel periphery of the magnetic bead. In some embodiments, the RNA template is conjugated before polymerization of the polyacrylamide. In some embodiments the RNA template is conjugated to the particle following polymerization of the polyacrylamide.

In some embodiments, the RNA template is a template for a protein or a peptide. In some embodiments, the RNA template is a template for a therapeutic protein or therapeutic candidate. In some embodiments, the RNA template is a template for a therapeutic target. In some embodiments, the RNA template is for an antibody, for example a single chain antibody. In some embodiments, the RNA template contains a sequence complementary to the gel bound oligonucleotides.

In some embodiments, the method comprises a translation system created by isolating, refining, and mixing factors involved in the translation-synthesis of proteins or peptides, such as a ribosome, translation factors, tRNAs, amino acids and energy sources including ATP and GTP.

Examples of natural tRNAs are mixtures of purified tRNA fractions obtained from collecting and crushing E. coli, which can also be obtained on the market. Some A, U, C and G in the natural tRNA are chemically modified by enzymes. Alternatively, tRNA having a naturally occurring sequence, albeit transcribed in the test tube, can also be used. In contrast, an artificial tRNA that is a transcription product of tRNA is preferably used as an orthogonal tRNA instead of natural tRNA. An artificial tRNA can be prepared by an in vitro transcription reaction using a template DNA and an appropriate RNA polymerase. Such artificial tRNAs do not include any chemical modification.

In some embodiments, the translation system is an E. coli S30 translation system (Promega, Madison, Wisconsin) for efficient in vitro translation. The E. coli S30 translation system provides advantageous high efficiency translation of a variety of mRNA templates, as compared to other in vitro translation systems (e.g., wheat germ extract, rabbit reticulocyte lysate). In addition, the S30 system for in vitro translation is well characterized and quite amenable to the preparation of very large reaction mixtures, thus facilitating the construction of very large libraries by the methods of the invention. The S30 system is also amenable to the incorporation of unnatural amino acids using tRNA molecules charged with unnatural amino acids. See PCT patent publication No. 90/05785, incorporated herein by reference. In some embodiments, the translation system is a synthetic mixture of recombinantly expressed and purified proteins involved in translation (e.g., PURExpress). These systems are advantageous as different components can be eliminated to drive the construction of certain types of libraries (e.g., elimination of release factors).

In some embodiments, the polypeptide encoded by the RNA comprises a detection tag. In some embodiments, the detection tag is an epitope recognized by an antibody or a sequence that can be bound by another protein. In some embodiments, the polypeptide comprises a HiBit, 6His, FLAG, HA, V5, streptavidin tag, GST tag, or MBP tag. In some embodiments, binding of the detection tag to a detection agent produces a detectable signal (e.g. fluorescence).

In some embodiments, the translated peptide or polypeptide is retained in the particle. in some embodiments, the translated peptide or polypeptide does not diffuse out of the particle. In some embodiments, the translated peptide or polypeptide is non-covalently bound to the particle or to a molecule associated with the particle. In some embodiments, peptide or polypeptide is covalently bound to the particle or a molecule associated with the particle. In some embodiments, the mRNA variously displays a puromycin tag that results in either covalent attachment or noncovalent attachment of the peptide to the mRNA.

In some embodiments, the RNA template encodes a peptide or polypeptide comprising multiple domains. For example, in some embodiments, the RNA template encodes a peptide or polypeptide of interest and a detection tag. In some embodiments, the RNA template encodes multiple detection tags.

In some embodiments, the domains are separated by a linker sequence. In some embodiments, the domains are separated by a flexible linker. In some embodiments, the linker sequence comprises glycines and serines. In some embodiments, the linker comprises the amino acid sequence (GₙS)_{X}, wherein n is 1 to 4 and x is 1 to 5. In some embodiments, the linker comprises the amino acid sequence G₄S (SEQ ID NO:19).

In some embodiments, the RNA template comprises one or more modifications. In some embodiments, the RNA template comprises a 3′ modification. In some embodiments, the RNA template comprises a 3′ puromycin-modified nucleotide.

In some embodiments, the translated peptide or polypeptide is about 5 to about 50, about 5 to about 40, about 5 to about 30, about 5 to about 20, or about 5 to about 10 amino acids in length.

In some embodiments, the method further comprises detecting and/or isolating particles with a desired property. In some embodiments, the particles are isolated using flow-cytometry, a cell sorting device, measuring a density, detecting an affinity tag, and/or detecting a DNA sequence. In some embodiments, fluorescence microscopy and flow cytometry are used to visualize specific epitope translation within particles.

In some embodiments, translated gel particles exhibit homogeneous antibody-derived fluorescence throughout the gel periphery while only magnetic bead autofluorescence are detected in the same particles before translation. In some embodiments, translated gel particle fluorescence via flow cytometry is baseline-separated compared to untranslated particles for selected epitopes, such as FLAG, HA, or V5.

In some embodiments, translation of gel-immobilized RNAs and subsequent immobilization of the translated products in the gel particle are verified by orthogonal detection of multiple epitope tags. In some embodiments, the translated products immobilized in the gel particle are peptides. In some embodiments, the translated products immobilized in the gel particle are therapeutic drugs. In some embodiments, immunofluorescence measurements are used to confirm the presence of each epitope of the immobilized or captured translation products in the gel particle by specific epitope labeling and in gel protein binding assays. In some embodiments, in-gel peptide capture yield is quantitated via HiBiT luminescence-based in vitro translation efficiency assay.

In some embodiments, the gel particle libraries prepared by the methods provided herein are screened via FACS. For example, in some embodiments, methods provided herein result in a gel particle library of peptide templates with degenerate codons fused to the HiBiT tag that are used to template magnetic beads via limiting dilution emulsion PCR (emPCR). In some embodiments, single bead qPCR analysis of the templated library beads indicates more than 4000 DNA templates/bead with about 30% of beads templated. In some embodiments, Library beads (5×10⁶) are combined with control epitope beads (e.g., 1% HA or V5), translated in the presence of puromycin oligonucleotide P4, probed with dye-labeled antibodies (e.g., anti-HA-647AF; anti-V5-CF488A), and sorted by FACS into two populations, i.e., high V5 and high HA. The sorted hit particles are sequenced to determine enrichment rates for the various control tags. Reads are pattern matched to epitope tag's sequences or the library's degenerate sequence. In some embodiments, the HA positive hit pool is 99% HA-encoding sequences and the V5 hit pool is 50% V5-encoding sequence, a 100-fold and 50-fold enrichment from the library starting material.

In some embodiments, the library-scale synthesis and sorting provided herein is compatible with engineered genetic codes. For example, in some embodiments, beads are functionalized with a DNA template that encodes a single codon (e.g., a native Gln codon) fused with the HiBiT tag and encapsulated in P1-functionalized gels. An engineered in vitro transcription and translation (IVTT) mix, which lacks glutamine and glutamine-tRNA synthetase (GlnRS) and contains noncanonical amino acid azidolysine (AzK) charged onto CUGERNA^{Asn}_{GGC} using the dFx flexizyme, is used to display the azide functionality via IVTT. In some embodiments, In some embodiments, the HiBit quantitation yields similar levels of captured peptide (105±7 nM) as the all-natural peptide epitopes captured in some other embodiments and examples provided herein. In some embodiments, a gel particle library of peptide templates with engineered codons fused to the HiBiT tag and displays AzK functionality is prepared using the methods described herein to result in an engineered IVTT mix.

In some embodiments, a peptide library template with 5NNT degenerate codons (excluding GAC codons) is used to template magnetic beads via emPCR, resulting in particles with an average of 2×10⁴ template molecules per bead. After gel encapsulation, the NNT library beads (3×10⁶) are combined with AzK templating beads (about 0.1% of beads) prepared herein and the in-gel transcription/translation is performed using the engineered IVTT mix with AzK functionality provided herein (no glutamine) and in the presence of P4. After translation, in-gel CuAAC click reaction is performed on the entire library mixture with 647AF-alkyne to label AzK presenting particles, then sorted the 647AF-high population (about top 2%) via FACS. After sequencing and decoding, an about 6-fold enrichment of GAC-templating sequences is observed in the sorted population compared to the starting library pool.

In some embodiments, provided herein are methods result in translation and detection of non-canonical amino acid-containing gel particle library beads. For example, in some embodiments, NNU library particles (3×10⁶) are subjected to an engineered in vitro translation (IVT) reaction that is provided to install an azido-lysine (AzK) at CUG codons. Translated particles are washed and treated with an AF647-alkyne in a CuAAC reaction before analysis by flow cytometry. The resulting particles are sorted to isolate the top 2% of the AF647 population and the sequences compared to the starting library. In some embodiments, a 6-fold enrichment is observed after one round of screening where the percentage of AzK sequences increases from about 0.09% in the original NNU library particles to about 0.58% after sorting and isolating the top 2% AF647 population using FACS.

In some embodiments, provided herein are standard PCR protocols for library generation, magnetic beads templated with several control epitope-coding sequences and doped into a conventional library as background being successfully sorted as hits and exhibited substantial enrichment compared to library input.

In some embodiments, provided herein are coupled transcription/translation of the mixed library/epitope particles without additional compartmentalization yielding sufficiently pure in-gel epitope display for immunofluorescence-based screening. The translation products, like the RNA transcripts, are concentrated in the gel matrix of the DNA-templated magnetic bead, showing that all enzymatic steps are proximity driven.

### VI. Kits and Libraries

In some embodiments, provided herein are kits for preparing a particle comprising a polymer coating and a magnetic core. In some embodiments, the kit comprises a monomer. In some embodiments, the kit comprises acrylamide and bis acrylamide. In some embodiments, the kit comprises one or more polymerization initiators. In some embodiments, the kit comprises an oil solution and an aqueous solution. In some embodiments, the kit comprises an oil solution comprising a polymerization initiator and an aqueous solution comprising a monomer. In some embodiments, the aqueous solution comprises magnetic beads. In some embodiments, the aqueous solution comprises an initiator. In some embodiments, the aqueous solution comprises TEMED. In some embodiments, the oil solution is in one container and the aqueous solution is in another contain. In some embodiments, emulsification of the oil solution with the aqueous solution results in polymerization of the monomer.

In some embodiments, the kit further comprises instructions for producing a particle according to the methods provided herein. In some embodiments, the kit comprises instructions for producing a library of particles provided herein.

Also provided herein are libraries of particles. In some embodiments, the library comprises particles with different oligonucleotide tags. In some embodiments, the library comprises particles with different building blocks. In some embodiments, the library comprises particles with different small molecules. In some embodiments, the library comprises particles with different mRNA sequences. In some embodiments, the library comprises particles with different peptides or polypeptides. In particular embodiments, the library includes between about 10² to 10¹⁰ particles (e.g., about 10² to 10³, 10² to 10⁴, 10² to 10⁵, 10² to 10⁶, 10² to 10⁷, 10² to 10⁸, 10² to 10⁹, 10³ to 10⁴, 10³ to 10⁵, 10³ to 10⁶, 10³ to 10⁷, 10³ to 10⁸, 10³ to 10⁹, 10³ to 10¹⁰, 10⁴ to 10⁵, 10⁴ to 10⁶, 10⁴ to 10⁷, 10⁴ to 10⁸, 10⁴ to 10⁹, 10⁴ to 10¹⁰, 10⁵ to 10⁶, 10⁵ to 10⁷, 10⁵ to 10⁸, 10⁵ to 10⁹, 10⁵ to 10¹⁰, 10⁶ to 10⁷, 10⁶ to 10⁸, 10⁶ to 10⁹, 10⁶ to 10¹⁰, 10⁷ to 10⁸, 10⁷ to 10⁹, 10⁷ to 10¹⁰, 10⁸ to 10⁹, 10⁸ to 10¹⁰, 10⁹ to 10¹⁰). In some embodiments, each particle comprises a different oligonucleotide, peptide, small molecule, polypeptide, or set of building blocks. In some embodiments, multiple particles within the library comprise the same oligonucleotide, peptide, small molecule, polypeptide, or set of building blocks. In some embodiments, each particle in the library comprises an identifying sequence that can be used to identify the set of building blocks, peptide, small molecule, or polypeptide.

### EXAMPLES

### Example 1—Diverse Functionalization of the Polyacrylamide Hydrogel Magnetic Particles Via Copolymerization

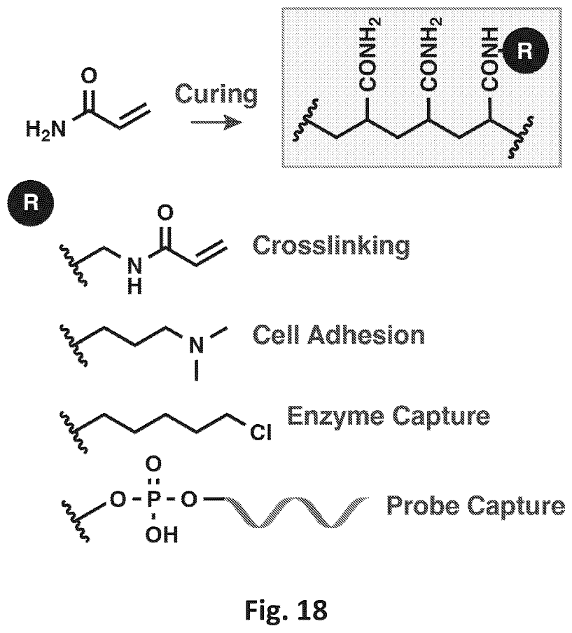

This example showed that diverse functionalization of the polyacrylamide hydrogel coated magnetic particles be achieved via copolymerization. The radical-mediated polymerization created the polyacrylamide hydrogel which incorporated a variety of functionalities to the polymer matrix by including molecules with both the desired functionality and a vinyl group for copolymerization. Various copolymerized functionalities were employed, including but not limited to crosslinkers, cell adhesion promoters (e.g., alkyl amines), affinity capture tags (e.g., chloroalkane HaloTag), enzyme capture, probe capture, and oligonucleotides for hybridization (FIG. 18). The latter two are useful for labeling and characterization.

Using this methodology, polyacrylamide hydrogels with both chemical (e.g., amine, azide) and biochemical (e.g., oligonucleotide) groups were synthesized. It was also demonstrated that these groups are able to participate in reactions in the hydrogel matrix of the particle system.

### Methods

Azido oligonucleotide preparation: Azide modification of 5′-NH₂ P1 DNA oligonucleotide primer with EDC, NHS, and 5-azidopentanoic acid to yield 5′-N₃ P1, followed by Cu(I)-catalyzed azide-alkyne cycloaddition (CuAAC) coupling of 5′-N₃ P1 to propargyl-modified magnetic microbeads (e.g., Dynabeads, 1.0 μm, 2.8 μm, or 10.0 μm dia) was conducted as previously reported (Malone et al. ACS Comb. Sci. 2017, 19, 1, 9-14).

Methacrylamide DNA Headpiece (ac-HDNA) functionalization: Methacrylic acid (100 μmol) was added to NHS (100 μmol) and EDC (100 μmol) in DMF and the esterification reaction was incubated for 5 min at room temperature. Methacrylic acid NHS ester solution (1 M, 15 μL) was added to HDNA (100 nmol) in phosphate buffer (0.4 M, pH 7.9), the reaction was incubated for 1.5 h at room temperature and then quenched with the addition of Tris buffer (20 μL, 1 M). The methacrylamide-HDNA (ac-HDNA) was purified by reversed-phase HPLC purification (Waters XTerra C18, 2.5 μm, 10 mm×50 mm) with gradient elution (mobile phase A: 97.5% 50 mM TEAA pH 8; mobile phase B: ACN; 2.5% B 1 min, 2.5-12.5% B 20 min, 4 mL/min) and absorbance detection (260 nm). Fractions containing the product of interest were pooled, dried and reconstituted in HPLC grade water for A260 quantitation. Fractions of interest were spotted (1 μL) on a MALDI-TOF MS target plate, dried, covered with THAP matrix solution (1 μL, 18 mg/mL THAP, 7 mg/mL ammonium citrate dibasic in 1:1 ACN:H2O), dried, and analyzed via MALDI-TOF MS (Microflex, Bruker Daltonics, Inc., Billerica, MA).

Hydrogel particle preparation: An aliquot of carboxylic acid-functionalized magnetic beads (M-270 carboxylic acid Dynabeads, 5×10⁷, ThermoFisher Scientific) was added to 1.5-mL microcentrifuge tube and isolated magnetically (FIG. 11). The supernatant was removed and the beads were suspended in acrylamide monomer solution (300 μL, 0.5 M 19:1, mono:bis-acrylamide, 0.6% APS). Oil (900 μL, Apr. 20, 1976 w/w/w, KF-6038/mineral oil/DMF-A-6cs) was layered on the bead suspension and the separated oil/aqueous sample was sparged with Ar (10 min). TEMED (1 μL) was added to the oil layer, the headspace in the tube was filled with Ar, and the sample was emulsified using a bead mill homogenizer (65 s, 2500 rpm, BeadBug, Benchmark Scientific, Sayreville, NJ), then incubated on ice (2 h, 4° C.). The polymerized gel particles were isolated magnetically, the supernatant was removed, and the particles were washed with breaking buffer (4×1 mL) until no trace of oil remained. Gel particles were suspended in bead buffer (1 mL) for storage. Gel particles were variously functionalized by adding the following reagents to the acrylamide monomer solution: propargyl methacrylate (20 μM, “alkyne” functionality), N-(3-aminopropyl) methacrylamide (0.002-20 mM, “amine” functionality), ac-P1 (20 μM, “reverse primer” functionality), or ac-HDNA (20 μM, HDNA functionality).

In-gel oligonucleotide hybridization: Gel particles were combined with FAM-P1′ (1 μM, 20 pmol per 1×10⁶ particles in 2×SSC, 0.5% SDS), where FAM-P1′ sequence is set forth in SEQ ID NO:4 and incubated for 2 min at room temperature. Gel particles were isolated magnetically, washed (2×200 μL 2×SSC, 0.5% SDS), and suspended in bead buffer (250 μL) for storage and analysis.

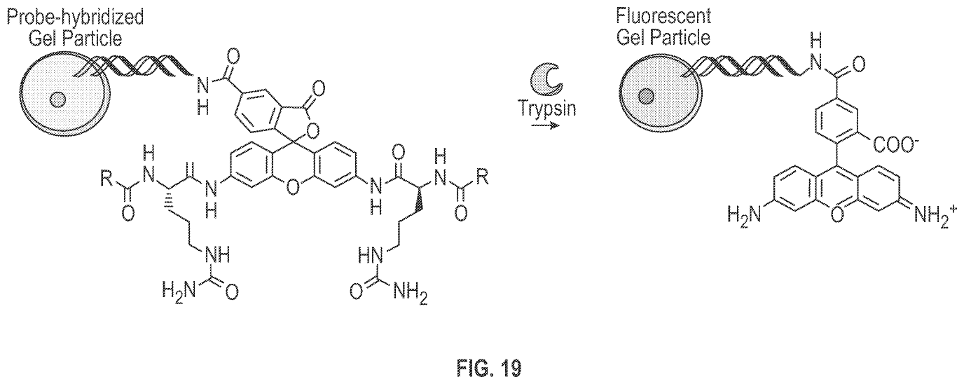

In-gel hybridization of an activity-based probe: Gel particles copolymerized with oligonucleotide and subsequently hybridized with a trypsin activity-based probe conjugated to the complementary oligonucleotide. The probe-hybridized gel beads were digested with trypsin, dequenching the activity-based probe and resulting in fluorescent gel particles (FIG. 19).

In-gel amine acylation: Gel particles (1× 10⁷) were suspended in phosphate buffer (0.2 M, pH 7.5, 100 μL), combined with NHS-647AF (1 nmol), and incubated for 1 h at room temperature. Gel particles were magnetically isolated, washed with bead buffer (2×200 μL), and suspended in bead buffer (200 μL) for storage and analysis.

In-gel CuAAC (Cu(I)-catalyzed azide-alkyne cycloaddition): Gel particles (1×10⁷) were suspended in reaction buffer (1 M TEAA pH 7, 0.5% Tween-20, 100 μL) and combined with N3-647AF (5 nmol). Catalyst mix (50 nmol CuSO4, 250 nmol ascorbic acid, 60 nmol THPTA) was added and the reaction was incubated for 1 h at room temperature. Gel particles were magnetically isolated, washed with bead buffer (2× 200 μL), and suspended in bead buffer (200 μL) for storage and analysis.

In-gel DNA Ligation: Gel particles (2×10⁶) copolymerized with ac-HDNA (20 μM) were suspended in T4 ligase buffer (20 μL, NEB) with enzymatic ligation substrate DNA oligonucleotides (L (+) and L (−), 50 μM each), heated (2 min, 95° C.) and cooled to room temperature. The thermally processed reaction mixture was split in half, T4 ligase (20 U, NEB) was added to one aliquot, and the samples were incubated for 1 h at room temperature. Gel particles were magnetically isolated, washed with bead buffer (2× 200 μL), and suspended in bead buffer (200 μL) for storage and analysis.

Confocal fluorescence imaging (particle analysis): Gel particles that were hybridized with FAM-P10′ were imaged via confocal fluorescence microscopy (Stellaris 8, Leica). Particles (˜10⁶) in buffer (10 mM Tris, 100 mM NaCl, 0.05% KF-6012) were loaded into an imaging well, and allowed to settle (15 min). Particle fluorescence (lₑₓ/lₑₘ=488/520 nm) was acquired and used to calculate object diameters (LAS).

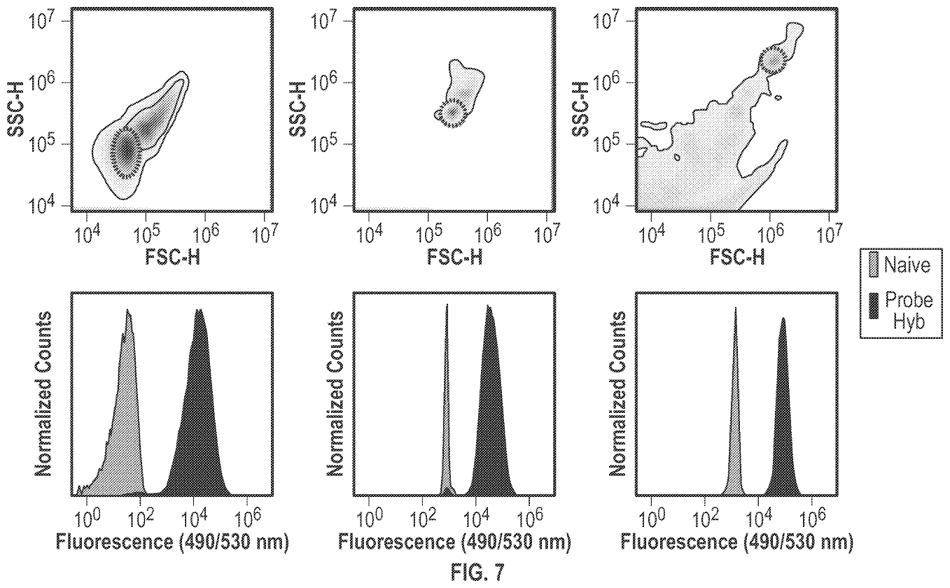

Flow cytometry (particle analysis): Gel particles were analyzed by flow cytometry (NovoCyte, Agilent). Gel particles were suspected in bead buffer (1-5×10⁴ beads/u L) for analysis. Particles were gated based on forward scatter (FSC) and side scatter (SSC) to isolate single particle populations (FIG. 7). Population fluorescence was reported for various channels (FAM λₑₓ/λₑₘ=488/530 nm; TMR λₑₓ/λₑₘ=561/580 nm; APC λₑₓ/λₑₘ=640/660 nm) based on the dye label used for gel particle probing.

### Results

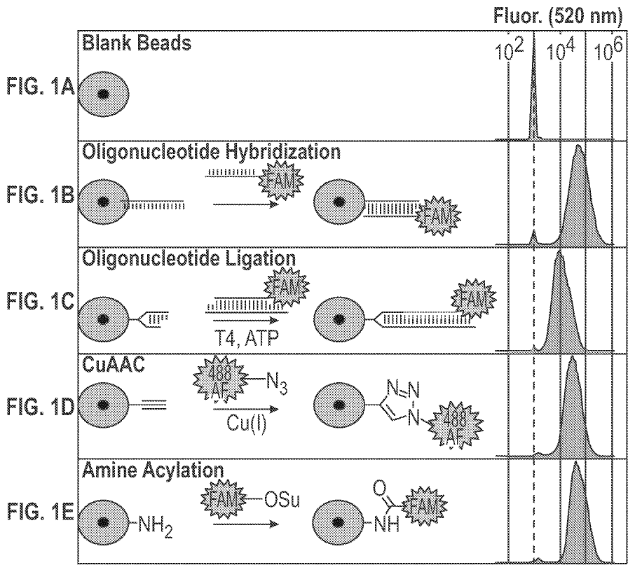

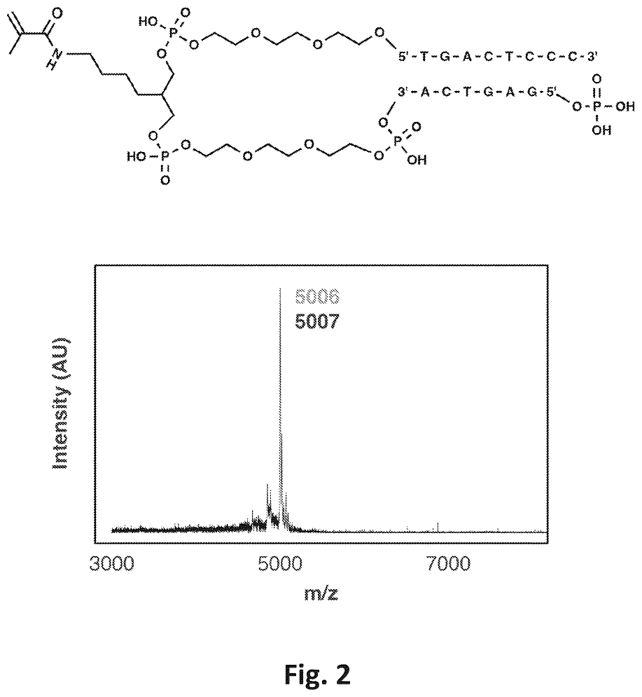

2.8-μm magnetic beads were encapsulated in hydrogels displaying diverse functionality (FIG. 1A-E). Specifically, oligonucleotides using either commercially available 5′-acrydite modification or amino oligonucleotides modified with methacrylic acid (FIG. 2), alkynes using propargyl methacrylate (PMA) (FIG. 1D), or primary amines using 3-(aminopropyl) methacrylamide (APMA) (FIG. 1E) were produced. Gel particles were copolymerized with 5′-methacrylamido oligonucleotide (FIG. 1B), methacrylamide-modified hairpin headpiece DNA (HDNA) (FIG. 1C), propargyl methacrylate (PMA) (FIG. 1D), or 3-(aminopropyl) methacrylamide (APMA) (20 μM additive) (FIG. 1E). Gel functionalization was detected via chemical reactions with fluorescent dye-labeled complementary functional groups, including complementary oligonucleotide hybridization (FIG. 1B), enzymatic ligation of dsDNA modules used for DEL synthesis (FIG. 1C), CuAAC with Alexa Fluor 488 azide (488AF-N3) (FIG. 1D), or amine acylation with fluorescein succinimidyl ester (FAM-OSu) (FIG. 1E). Gel labeling was detected via flow cytometry and compared to blank gel beads (FIG. 1, right).

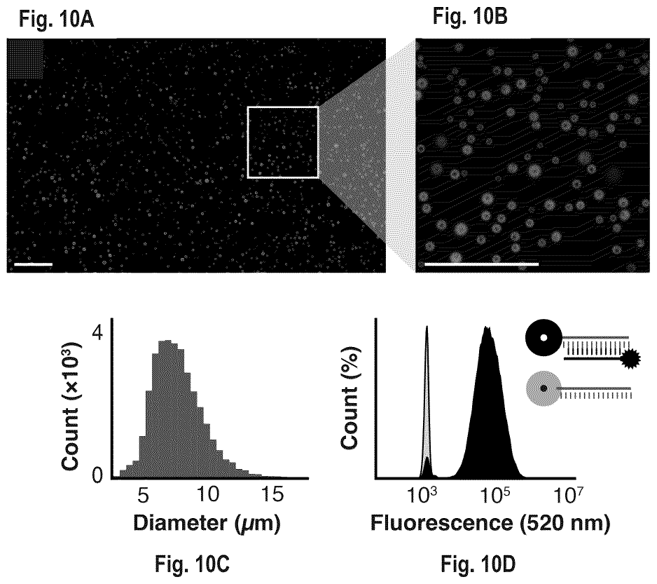

Copolymerization with a 5′-methacrylamide-modified DNA oligonucleotide P1 (acrydite) yielded P1-functionalized gel particles, which were hybridized with a 5′-fluorescein (FAM)-labeled DNA oligonucleotide complementary to P1 (FAM-P1′) and analyzed by confocal fluorescence imaging (FIGS. 10A-10B). Over nine preparations (32000 sampled particles), a median diameter of 7±2 μm (FIG. 10C) was observed. Flow cytometry analysis of the FAM-P1′-probed gel particles yielded a distinct population of signals that were 100-fold increased in fluorescence intensity compared to unprobed gel particles and indicated that >95% of magnetic beads were encapsulated in hydrogel (FIG. 10D).

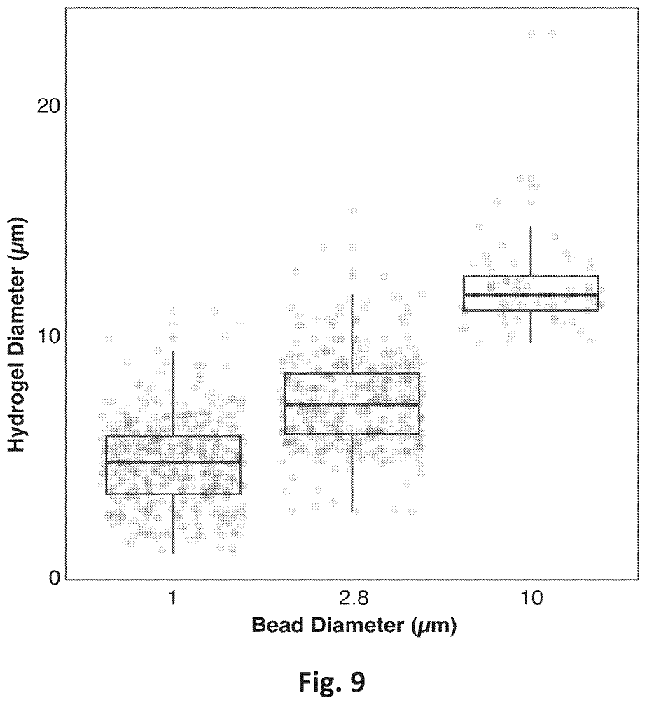

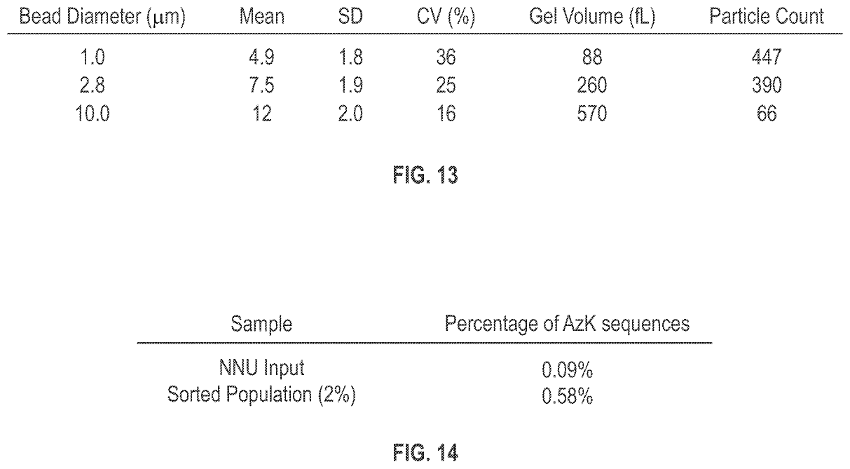

Increasing the magnetic bead size formed larger and more uniform gel particle distributions (FIG. 13) by imaging microscopy (FIG. 9), and all bead sizes (e.g., 1.0, 2.8, and 10.0 μm diameter magnetic beads encapsulated in hydrogel respectively) yielded populations readily gated by forward and side scatter in flow cytometry for comparison of hydrogel fluorescence upon probing (FIG. 7).

The particle functionalization was detected and characterized by flow cytometry with the addition of the appropriate fluorescent dye-labeled substrates, including complementary oligonucleotide, double-stranded oligonucleotide ligation module, azide, or succinimidyl ester, respectively. Depending on reactivity, the relative fluorescence increased between 5 and 25-fold compared to naive gel particles (FIG. 1, right). Hybridization (FIG. 1B, right) and amine acylation (FIG. 1E, right) resulted in the largest shift; all functionalization reactions resulted in baseline separation of product and starting material by flow cytometry (FIG. 1, right).

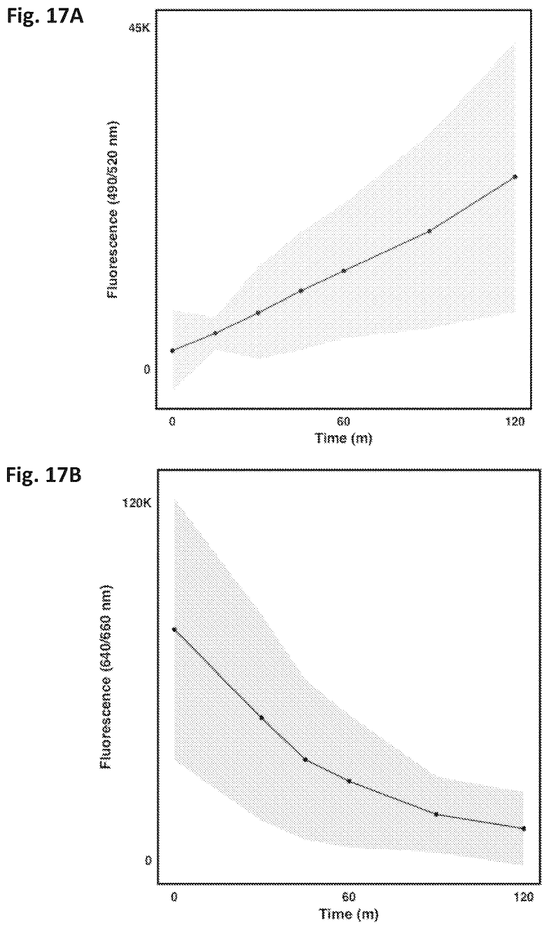

The hydrogel particles were also tested to perform enzyme activity-based assay. For example, the hydrogel particles were first copolymerized with oligonucleotide and subsequently hybridized with a trypsin activity-based probe conjugated to the complementary oligonucleotide. The probe-hybridized gel beads were then digested with trypsin, dequenching the activity-based probe and resulting in fluorescent gel particles. FIG. 17A is the time-dependent quantitation of hydrogel particles loaded with the fluorogenic (turn-on) green fluorescent probe of trypsin activity. FIG. 17B is the time-dependent quantitation of hydrogel particles loaded with an N-terminally-labeled tryptic peptide, wherein the N-terminal label is a red-fluorescent dye, Cy5. Upon tryptic digestion, Cy5 is liberated from the gel, resulting in a decrease in fluorescence signal over time (turn-off).

Hydrogel particles with escalating amine loading capacity were prepared by copolymerizing increasing concentrations of APMA (0.02-20 mM); acylation with FAM-OSu resulted in log-linear increases in gel particle fluorescence over the 4 order of magnitude titration (FIG. 3A, B).

Hydrogel particles were diversely functionalized by including sub-stoichiometric methacrylamide-modified additives in the acrylamide: bis-acrylamide monomer solution for copolymerization. Incorporation of the synthetic hairpin headpiece DNA (HDNA) and subsequent proof-of-concept enzymatic oligonucleotide ligation reaction demonstrated the viability of standard DEL synthesis workflows using these particles.

The particle loading capacity of amine functionality was demonstrated to be quantitative over 4 orders of magnitude. At the highest site density (20 mM), each median particle (7 μm in diameter) particle harbored ˜4 fmol amine sites.

### Conclusions

In this example, magnetic core hydrogel particles successfully supported diverse functionalization via copolymerization and subsequent chemical and biochemical solid-phase synthesis-like procedures. Radical-mediated polymerization is mild and amenable to incorporating a variety of acryloyl and methacryloyl moieties. This example demonstrated some of the most common functionalities deployed in chemical library synthesis, but the technology can be applied to many other functionalities. This example should not be construed as limiting the scope of the invention.

In addition, the time dependent fluorescence data (FIG. 17A, B) demonstrates the utility of the hydrogel scaffold to facilitate biochemical enzyme activity assays. The in-gel assay format can be used to detect, for example, the presence of an inhibitory element that is also synthesized in the gel (e.g., a DNA-encoded library member, an aptamer, a peptide). The in-gel assay can also be used to probe for catalytic activity in an enzyme engineering experiment wherein each particle contains nucleic acid templates for in vitro translation.

### Example 2—in-Gel Transcription of DNA and Subsequent Capture of the Resulting mRNA Via Hybridization in the Hydrogel Periphery of the Magnetic Bead

While enzymatic ligation plays an important role in the preparation of DNA encoded chemical libraries, DNA transcription is central in the preparation of genetically-encoded RNA and protein libraries. This example shows that RNA transcribed from the bead-bound DNA templates is captured in the peripheral hydrogel by a complementary RNA sequence in the hydrogel.

### Methods

Bead templating by PCR (single templates): P1-functionalized magnetic microbeads (1×10⁸) were suspended in PCR mix (0.4 mM dNTPs, 4 μM forward primer P2, 0.1 U/μL Taq DNA polymerase in 1×standard Taq buffer) containing DNA template (1 pmol/μL) and thermally cycled ([95° C., 20 s; 60° C., 20 s; 68° C., 20 s]×25 cycles, C1000 Touch, Bio-Rad). Beads were washed with bead buffer (4×1 mL) and suspended in bead buffer (1 mL) for analysis.

qPCR mix (0.2 mM each dNTP, 0.5 μM each P1 and P2 primers, 0.25 μM qPCR probe, where qPCR probe sequence is set forth in SEQ ID NO:7, 0.05 U/μL Taq polymerase, 1×standard Taq buffer) was prepared and aliquoted (20 μL each) to a 96-well PCR plate. Dilutions (1/100 and 1/1000) of templated bead samples were prepared in bead buffer. qPCR wells were assembled by adding diluted suspensions (1 μL). The reactions were thermally cycled ([95° C., 20 s; 60° C., 20 s; 68° C., 20 s]×40 cycles), monitoring fluorescence (530 nm, QuantStudio3, Thermo Scientific). A standard curve was prepared using serial dilutions of the template, adding a constant volume (1 μL) to each standard reaction (100 pg/μL-0.1 fg/μL in logs). Beads were counted by hemocytometer to obtain the average per bead template load.

In-gel Transcription: Untemplated beads labeled with 647AF and beads templated with DNA encoding the FLAG epitope (FLAG-templated beads) were encapsulated in hydrogels copolymerized with P1 (50 μM) in separate emulsion polymerizations. Cured gel particles (1× 10⁷ each untemplated and FLAG-templated) were suspended in T7 RNAP reaction mix (0.5 mM NTPs, 5 mM DTT, 5 U/μL T7 RNAP, 0.4 U/μL TIPP in 1×NEB T7 buffer, 1 μM P3) and incubated (1 h, 37° C.). Particles were washed with breaking buffer (2×500 μL) and suspended in bead buffer (400 μL) for storage and analysis.

Flow cytometry (particle analysis): Gel particles were analyzed by flow cytometry (NovoCyte, Agilent). Gel particles were suspended in bead buffer (1-5×10⁴ beads/μL) for analysis. Particles were gated based on forward scatter (FSC) and side scatter (SSC) to isolate single particle populations (FIG. 7). Population fluorescence was reported for various channels (FAM λₑₓ/λₑₘ=488/530 nm; TMR λₑₓ/λₑₘ=561/580 nm; APC λₑₓ/λₑₘ=640/660 nm) based on the dye label used for gel particle probing.

### Results

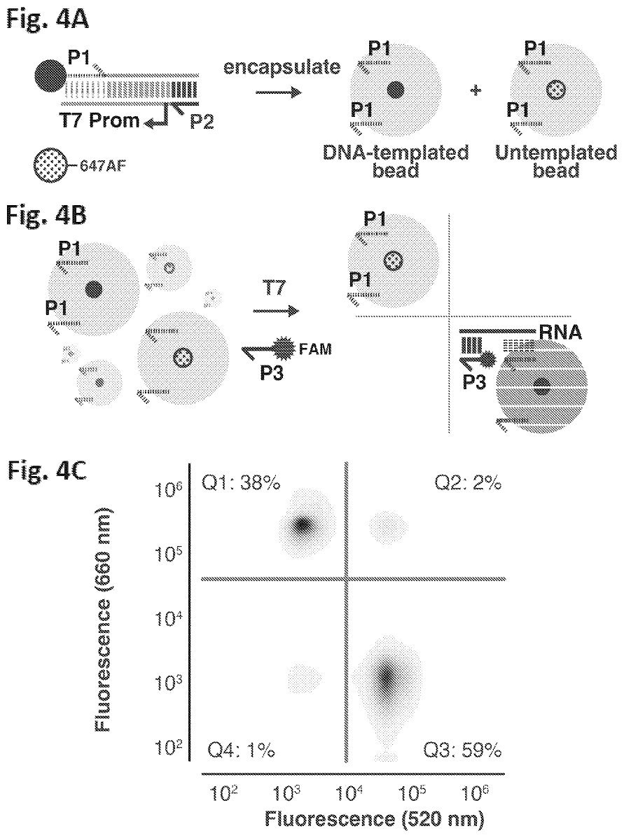

DNA-templated 2.8-μm magnetic beads were mixed with untemplated, dye-labeled (Alexa Fluor 647, 647AF) beads as negative controls (FIG. 4A). RNA transcripts were detected by including a FAM-labeled DNA oligonucleotide probe of the RNA transcript 5′ region (P3). (FIG. 4B). After in vitro transcription of the bead mixture, flow cytometry analysis indicated the presence of 3 populations of beads: 38% high 647AF fluorescence (i.e., the untemplated negative control beads), 59% high FAM fluorescence (i.e., the DNA-templated beads), and 2% fluorescent in both channels (FIG. 4C).

DNA-templated beads were prepared by PCR using DNA oligonucleotide primer P1 (FIG. 4A)-functionalized magnetic beads (FIG. 4A) and DNA oligonucleotide primer P2 (FIG. 4A). DNA templates contained a T7 RNA polymerase promoter element (T7 prom, FIG. 4A). Untemplated negative control beads were labeled with Alexa Fluor 647 (647AF, FIG. 4A). Each bead set was encapsulated in P2-copolymerized hydrogel. In vitro transcription of the gel-encapsulated DNA-templated and untemplated beads in the presence of FAM-labeled DNA oligonucleotide probe of the RNA transcript 5′ region (FIG. 4B, P3) detected the presence of RNA transcript hybridized via P2 in the gel. Two-dimensional flow cytometry analysis showed that the majority of particles exhibited either exclusively red fluorescence (38%) (FIG. 4C, 660 nm, untemplated negative controls in Q1) or green fluorescence (59%) (FIG. 4C, 520 nm, templated and RNA-loaded beads in Q3).

### Conclusions

The flow cytometry results show that RNA was captured by hybridization in the hydrogel periphery of the magnetic bead. The results also showed that migration of transcripts onto untemplated beads not only occurred infrequently but as a distinct population. Therefore, due to the low levels of migration of transcripts, this method is amenable to production of libraries with large amounts of different templates.

### Example 3—in-Gel Translation of Peptides and Capture of the Translated Peptides in the Hydrogel Periphery of the Magnetic Beads

This example demonstrated the in-gel translation of peptides and subsequent capture or immobilization of the peptide products in the hydrogel periphery of the magnetic beads.

### Methods

In-gel Translation (biogenic amino acids): DNA-templated beads were encapsulated in hydrogels copolymerized with ac-P1 (50 μM) and acrylamide-modified BSA (50 μM). BSA (2 μmol) N-acryloxysuccinimide (20 μmol) were combined in buffer (20 mM phosphate pH 7.5, 20 μL) and incubated for 1 h at room temperature. Hydrogel particles (1×10⁵ particles/μL, 10-70 μL final volume) were suspended in NEB PURExpress® ΔRF reaction mix (RF1 omitted) containing puromycin capture oligonucleotide P4 (10 μM). Reactions were incubated (3 h, 37° C.), washed with breaking buffer (0.5 mL), washed with PBST (0.5 mL), and suspended in PBST (10⁴ particles/μL).

In-gel Translation (unnatural amino acids): Reagents and protocols for the in vitro translation of noncanonical amino acids were adapted from previously described methods (Adaligil et al. ACS Chem. Biol. 2021, 16, 6, 1011-1018, Murakami et al. Nature Methods 2006, 3, 357-359).

In-gel immunofluorescence detection: Translated particles were suspended in detection antibody solution (4×104 particles/μL, 10 ng/μL APC anti-HA, 50 ng/μL CF488 anti-V5, 10% non-fat milk in PBST), incubated (16 h, 4° C.), washed with PBST (0.5 mL) and suspended in PBST (1 mL) for flow cytometry.

Flow cytometry (particle analysis): Gel particles were analyzed by flow cytometry (NovoCyte, Agilent). Gel particles were suspected in bead buffer (1-5×104 beads/u L) for analysis. Particles were gated based on forward scatter (FSC) and side scatter (SSC) to isolate single particle populations (FIG. S2). Population fluorescence was reported for various channels (FAM λₑₓ/λₑₘ=488/530 nm; TMR λₑₓ/λₑₘ=561/580 nm; APC λex/λₑₘ=640/660 nm) based on the dye label used for gel particle probing.

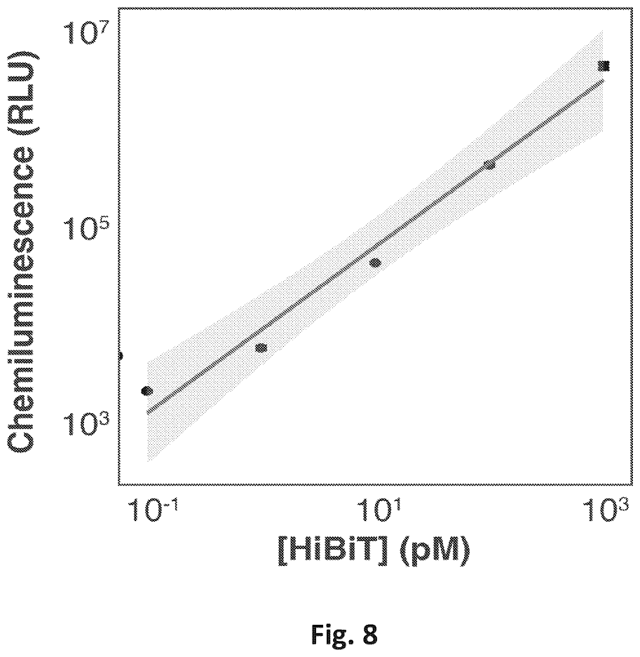

HiBIT Assay: Sample (5 μL solution or 2.5×10⁴ hydrogels in 5 μL PBS) was combined with LgBiT/substrate mixture (5 μL, 2×LgBiT/substrate in PBS) in a microtiter well plate (black, 384-well), and incubated (15 min, 37° C.). HiBiT peptide standard solutions (0.1-1000 nM HiBiT peptide) were similarly assembled and incubated. Luminescence was analyzed via plate reader (CLARIOstar Plus, BMG LABTECH) and unknown concentrations determined based on HiBiT standard analysis (FIG. 8).

### Results

Magnetic beads were templated with DNA encoding various affinity tag epitopes (FLAG, HA, or V5) fused to a HiBiT luciferase complementation tag and encapsulated in P1-functionalized gels. The gel particles were subjected to mRNA display-type in vitro transcription/translation reactions incorporating a puromycin-modified peptide capture oligonucleotide P4 complementary to the RNA directly 3′ of the stop codon (FIG. 5A). Translated beads were analyzed by imaging microscopy and flow cytometry to visualize specific epitope translation. Translated gel particles exhibited homogeneous antibody-derived fluorescence throughout the gel periphery (FIGS. 5B-5C); only magnetic bead autofluorescence was detected in the same particles before translation (FIG. 5D). Translated gel particle fluorescence via flow cytometry was baseline-separated compared to untranslated particles for all three example epitopes (FIG. 5F). In-gel peptide capture yield quantitated via HiBiT luminescence. Particles translated in the presence of puromycin capture oligonucleotide P4 retained ˜100 nM HiBiT peptide, while <1 nM was retained in translations lacking P4 (FIG. 5G).

### Conclusions

Orthogonal detection of multiple epitope tags in this example successfully demonstrated the translation of gel-immobilized RNAs and subsequent capture or immobilization of the translation product in the gel particle. Immunofluorescence measurements confirmed the presence of each epitope by specific epitope labeling, also demonstrated proof-of-concept for conducting in-gel protein binding assays. HiBiT detection additionally confirmed the presence of captured peptide and enabled high-sensitivity quantitation of captured peptide. The in-gel peptide capture yields (˜100 nM) were sufficient for robust detection in both immunofluorescence and HiBiT assays.

### Example 4—the Polyacrylamide Hydrogel Magnetic Particle's Compatibility with FACS Analysis as a High-Throughput Screening Strategy

This example demonstrated the novel gel particle format's utility by showing its compatibility with FACS analysis as a high-throughput screening strategy. In addition, the data also demonstrated that library-scale synthesis and sorting is compatible with engineered genetic codes.

### Methods

Bead templating by PCR (single templates): P1-functionalized magnetic microbeads (1×10⁸) were suspended in PCR mix (0.4 mM dNTPs, 4 μM forward primer P2, 0.1 U/μL Taq DNA polymerase in 1×standard Taq buffer) containing DNA template (1 pmol/μL) and thermally cycled ([95° C., 20 s; 60° C., 20 s; 68° C., 20 s]×25 cycles, C1000 Touch, Bio-Rad). Beads were washed with bead buffer (4×1 mL) and suspended in bead buffer (1 mL) for analysis.

qPCR mix (0.2 mM each dNTP. 0.5 μM each P1 and P2 primers, 0.25 μM qPCR probe, 0.05 U/μL Taq polymerase, 1×standard Taq buffer) was prepared and aliquoted (20 μL each) to a 96-well PCR plate. Dilutions (1/100 and 1/1000) of templated bead samples were prepared in bead buffer. qPCR wells were assembled by adding diluted suspensions (1 μL). The reactions were thermally cycled ([95° C., 20 s; 60° C., 20 s; 68° C., 20 s]×40 cycles), monitoring fluorescence (530 nm, QuantStudio3, Thermo Scientific). A standard curve was prepared using serial dilutions of the template, adding a constant volume (1 μL) to each standard reaction (100 pg/μL-0.1 fg/μL in logs). Beads were counted by hemocytometer to obtain the average per bead template load.

Bead templating by emPCR (NNK library templates): P1-functionalized magnetic microbeads (1×10⁸) were suspended in PCR mix (0.2 mM each dNTP, 8 μM forward primer P2, 0.02% w/v KF-6102, 0.3 U/μL Taq DNA polymerase in 1×standard Taq buffer) containing NNK₅ DNA template (1.2 fg/μL). Oil (900 μL, Apr. 20, 1976, KF-6038/mineral oil/DMF-A-6CS, w/w/w) was added to the top of each aqueous reaction mix. The reaction was emulsified (65 s, 2500 rpm) using a bead mill homogenizer (BeadBug, Benchmark Scientific). Using a wide bore pipet tip, aliquots (50 μL) were transferred to a 96-well PCR plate and the samples were thermally cycled ([95° C., 20 s; 60° C., 20 s; 68° C., 30 s]×35 cycles, 68° C. 5 min). The plate was placed on a magnet stand and incubated (30 min), the supernatant was removed, the isolated beads were transferred in breaking buffer to a clean 1.5-mL tube, and washed with breaking buffer (4×1 mL). Washed beads were isolated and suspended in bead buffer (1 mL) for analysis.

qPCR mix (0.2 mM each dNTP, 0.5 μM each P1 and P2 primers, 0.25 μM qPCR probe, 0.05 U/μL Taq polymerase, 1×standard Taq buffer) was prepared and aliquoted (20 μL each) to a 96-well PCR plate. Beads were counted by hemocytometer to prepare suspensions in bead buffer of known densities (100 beads/u L and 1 bead/μL). Three 100-bead and 77 single-bead qPCR wells were assembled by adding appropriate suspension (1 μL). The reactions were thermally cycled ([95° C., 20 s; 60° C., 20 s; 68° C., 20 s]×40 cycles), monitoring fluorescence (530 nm, QuantStudio3). A standard curve was prepared using serial dilutions of the template, adding a constant volume (1 μL) to each standard reaction (100 pg/μL-0.1 fg/μL in logs).

Flow cytometry (library screening): Gel particles were screened by FACS (FACSAria III, BD Biosciences). Single particle populations (gated by FSC/SSC correlation as described above) were analyzed for multi-spectral fluorescence in various channels (FAM λex/λem=488/530 nm; APC λₑₓ/λₑₘ=640/60 nm) based on the dye label used for gel particle probing.

In-gel immunofluorescence detection: Translated particles were suspended in detection antibody solution (4×104 particles/μL, 10 ng/μL APC anti-HA, 50 ng/μL CF488 anti-V5, 10% non-fat milk in PBST), incubated (16 h, 4° C.), washed with PBST (0.5 mL) and suspended in PBST (1 mL) for flow cytometry.

NGS Library Preparation & Analysis: Bead aliquots (˜1,000 beads) from each of the flow cytometry sorted populations were amplified using primers P1 and P2 and gel purified to isolate the amplicons within the range of the templates (180-220 bp for all samples). Illumina sequencing libraries were constructed using the Bioo Scientific NEXTflex Rapid DNA-Seq kit and NEXTflex unique dual index DNA barcodes (Bioo Scientific Corporation, Austin TX). Five nanograms of amplicon sample was taken into end repair, adenylation and adapter ligation reactions. The adapter ligated product was cleaned up without library size selection using Agencourt AMPure XP beads (Beckman Coulter, Inc. Brea, CA). The cleaned ligated DNA was amplified by 10 PCR cycles to enrich for adapter ligated products. The amplified PCR product was cleaned by AMPure XP beads and quantified by Kapa qPCR (Kapa Biosystems, Inc. Wilmington, MA). The libraries were denatured and diluted to 12 pM for clustering on the MiSeq (Illumina Inc., San Diego, CA) using v2 Micro SR 300 cycles chemistry and dual indexing. Analysis was performed by aligning each sequence to the control epitope or library sequences and counting the proportion of matches (maximum of 3 mismatches) to each aligned sequence.

Synthesis of aminoacyl-azidolysine-CUG-tRNA^{Asn}: Aminoacylation reactions (20 μL, 20 μM tRNA and 20 μM dFx flexizyme in 0.1 M Bicine, pH 9.0) were heated (95° C., 3 min) and cooled to room temperature over 5 min. 20 mM MgCl₂ was added and the mixture was chilled on ice (5 min). The reaction was initiated by addition of azidolysine 3,5-dinitrobenzyl ester (25 mM in DMSO) and incubated on ice for 2 hr. After the acylation reaction, the aminoacyl-N-e-azidolysine-CUG-tRNA^{Asn} was precipitated (0.3 M NaOAc, pH 5.2, 100% EtOH, 10,000 g, 15 m). The pellet was rinsed (0.1 M NaOAc, pH 5.2, 70% EtOH) and dried.

In vitro translation of AzK-containing peptides: N-e-azidolysine-containing peptides were translated in an genetically reprogrammed in vitro translation system from recombinant E. coli, with glutamine and GlnRS omitted to reprogram N-e-azidolysine to the GAC codon. Briefly, in vitro translation reactions contained 50 mM HEPES pH 7.6, Mg(OAc) 2, 100 mM KOAc, 1 mM DTT, 2 mM spermidine, 20 mM creatine phosphate, 2 mM ATP, 2 mM GTP, 1 mM CTP, 1 mM UTP, 0.2 mM each amino acid, and 1.5 mg/mL E. coli total tRNA with final protein concentrations of 0.03 μM ArgRS, 0.09 μM GlyRS, 0.02 μM HisRS, 0.4 μM IleRS, 0.02 UM LeuRS, 0.11 μM LysRS, 0.68 μM PheRS, 0.04 UM SerRS, and 0.02 μM ValRS, 0.6 μM MTF, 2.7 μM IF1, 0.4 μM IF2, 1.5 μM IF3, 0.26 μM EF-G, 10 μM EF-Tu/Ts, 5 μM EF-P. 0.25 μM RF2, 0.17 μM RF3, 0.5 μM RRF, 1 μM T7 RNA polymerase, 3 μg/mL MK, 4 μg/mL creatine kinase, and 1.2 μM ribosome. Hydrogel particles (10⁵ particles/μL, 10-30 μL final volume) were suspended in the in vitro translation mix containing puromycin capture oligonucleotide P4 (10 μM) and N-e-azidolysine-CUG-RNA^{Asn}. Reactions were incubated (3 h, 37° C.), washed with breaking buffer (0.5 mL), washed with PBST (0.5 mL), and suspended in PBST (10⁴ particles/μL) for analysis.

Library-scale Synthesis and Sorting with Engineered Genetic Codes: Beads were functionalized with a DNA template that encodes a single GAC codon (which natively encodes Gln) along with the HiBiT tag and encapsulated in P1-functionalized gels. An engineered IVT mix was used, where glutamine was excluded and GlnRS charged the noncanonical amino acid azidolysine (AzK) onto CUGtRNAAsn using the dFx flexizyme, allowing the specific display of the azide functionality using in vitro translation.

### Results

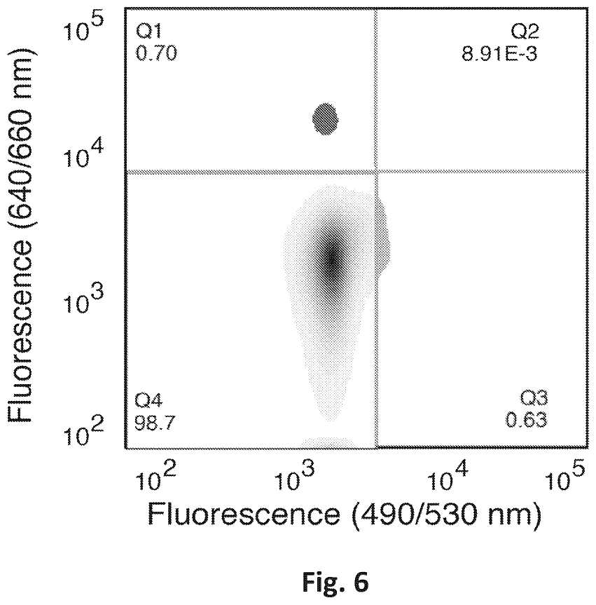

A peptide library template with 5 NNK degenerate codons fused to the HiBiT tag was used to template magnetic beads via limiting dilution emPCR. Single-bead qPCR analysis of the templated library beads indicated 4.2k DNA templates/bead with ˜30% of beads templated. Library beads (5×10⁶) were combined with control epitope beads (1% HA or V5), translated in the presence of puromycin oligonucleotide P4, probed with dye-labeled antibodies (anti-HA-647AF; anti-V5—CF488), and sorted by FACS into two populations (high V5 & high HA; FIG. 6).

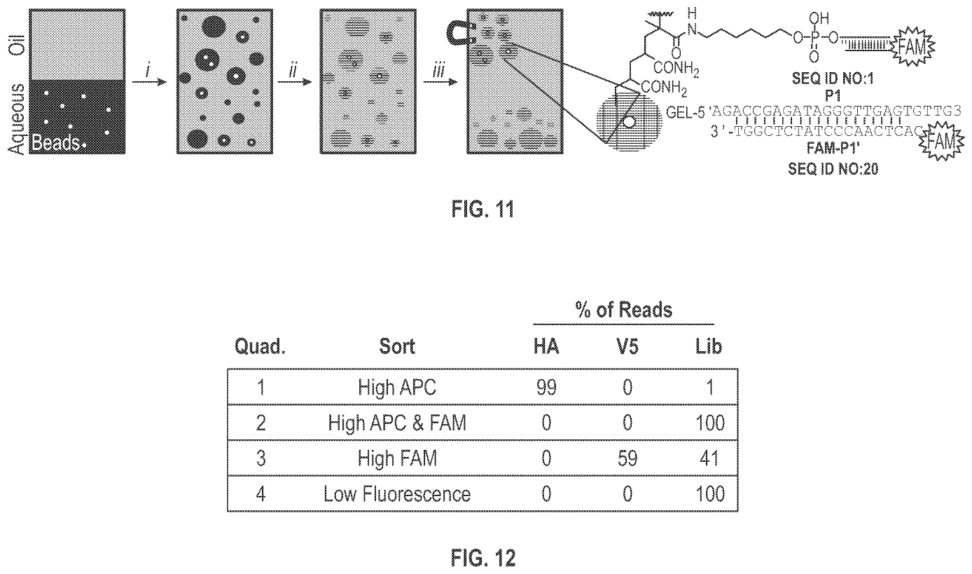

The sorted hit particles were sequenced to determine enrichment rates for the various control tags. Reads were pattern matched to epitope tag sequences or the NNK₅ library degenerate sequence. The HA positive hit pool was ˜99% HA-encoding sequences and the V5 hit pool was ˜50% V5-encoding sequence, a ˜100-fold and ˜50-fold enrichment from the library starting material (FIG. 12).

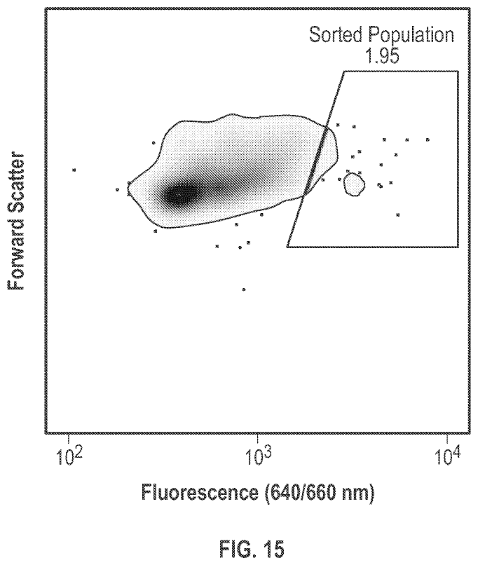

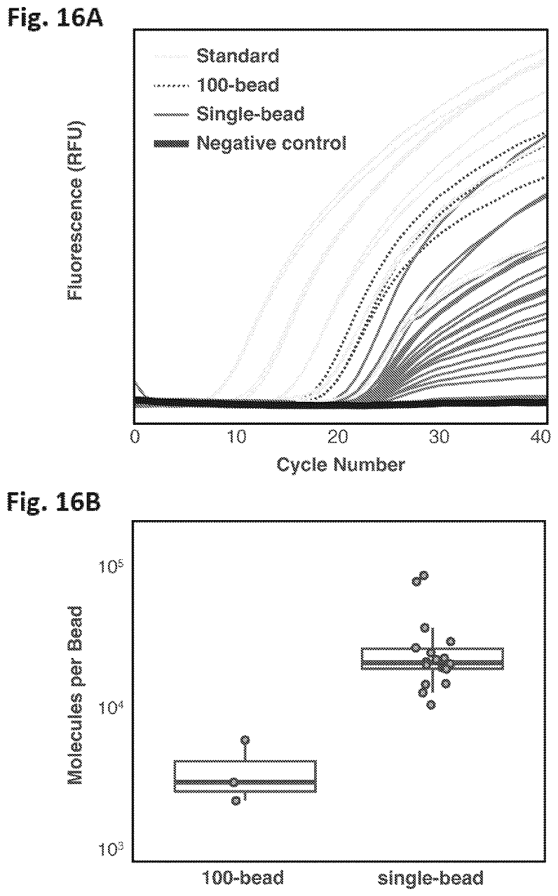

The data also established that library-scale synthesis and sorting is compatible with engineered genetic codes. HiBit quantitation yielded similar levels of captured peptide (105±7 nM) as the all-natural peptide epitopes previously tested (FIG. 5G). A peptide library template with 5 NNT degenerate codons (to exclude GAC codons) was designed and encapsulated magnetic beads bearing these sequences (bead QC) were generated. The NNT library beads (3×10⁶) were combined with a small amount of our AzK templating beads (0.1% of beads), where Azidolysine (AzK) peptide-encoding sequence is set forth in SEQ ID NO: 18, and were performed translation using the engineered IVT mix in the presence of P4. After translation, the in-gel CuAAC was performed on the entire library mixture with 647AF-alkyne to label any AzK-presenting particles and sorted the 647AF-high population (top 2%) via FACS (FIG. 15). After high throughput sequencing and decoding, an about 6-fold enrichment of CUG-templating sequences among the sorted population compared to the starting library pool was observed (Table 2) and (FIG. 14).

### Conclusions

These experiments in this example established the feasibility of FACS-based high-throughput screening of gel particle libraries. Using standard PCR protocols for library generation, magnetic beads templated with several control epitope-coding sequences and doped into a conventional library as background were successfully sorted as hits and exhibited substantial enrichment compared to library input. Importantly, coupled transcription/translation of the mixed library/epitope particles without additional compartmentalization yielded sufficiently pure in-gel epitope display for immunofluorescence-based screening. Translation products, like RNA transcripts, were concentrated in the gel periphery of the DNA-templated magnetic bead.

TABLE 1
Oligonucleotide Sequences

| Name | Description | Sequence (5′-3′) | SEQ ID NO |
|---|---|---|---|
| P1 | reverse primer | AGACCGAGATAGGGTTGA | SEQ ID NO: 1 |
| | | GTGTTG | |
| ac-P1 | acrydite P1 | /5Acryd/AGACCGAGATAGG | SEQ ID NO: 2 |
| | | GTTGAGTGTTG | |
| P2 | forward primer | TGCGTCCGGCGTAGAGGAT | SEQ ID NO: 3 |
| | | C | |
| FAM-P1′ | P1 probe | /56- | SEQ ID NO: 4 |
| | | FAM/CACTCAACCCTATCTC | |
| P3 | mRNA probe | /56- | SEQ ID NO: 5 |
| | | FAM/CTTGTCGTCATCGTCT | |
| | | TTGTAGTC | |
| P4 | puromycin linker | GTGTTGCCGCCCCCCGTC/iS | SEQ ID NO: 6 |
| | | p18//iSp18//iSp18//3Puro/ | |
| qPCR probe | qPCR probe | /56-FAM/TATATCTCC/ZEN/ | SEQ ID NO: 7 |
| | | TTCTTAAAGTTAACCCTAT | |
| | | AGTGAGTCG/3IABKFQ/ | |
| HDNA | headpiece DNA | /5Phos/GAGTCA/iSp9// | SEQ ID NO: 8 |
| | | iUniAmM//iSp9/TGACTCCC | |
| L(+) | ligation (+) | GGGCCGCC/iFluorT/TCGTCC | SEQ ID NO: 9 |
| | | TTCTCAGCGAC | |
| L(−) | ligation (−) | /5Phos/CCATGTCGCTGAGA | SEQ ID NO: 10 |
| | | AGGACGAAGGCGGCCCGG | |
| V5 | V5 (affinity tag | TGCGTCCGGCGTAGAGGAT | SEQ ID NO: 11 |
| | epitope)-encoding | CCTAGTAATACGACTCACT | |
| | sequence | ATAGGGTTAACTTTAAGAA | |
| | | GGAGATATACATATGGGTA | |
| | | AGCCTATCCCTAACCCTCT | |
| | | CCTCGGTCTCGATTCTACG | |
| | | GGAGGTGGTGGAAGTGTCA | |
| | | GCGGCTGGCGGCTGTTCAA | |
| | | GAAAATCAGTTAGGACGG | |
| | | GGGGCGGCAACACGGCTC | |
| | | GAGCAACACTCAACCCTAT | |
| | | CTCGGTCT | |
| HA | HA (affinity tag | TGCGTCCGGCGTAGAGGAT | SEQ ID NO: 12 |
| | epitope)-encoding | CCTAGTAATACGACTCACT | |
| | sequence | ATAGGGTTAACTTTAAGAA | |
| | | GGAGATATACATATGTACC | |
| | | CATACGATGTTCCAGATTA | |
| | | CGCTGGAGGTGGTGGAAGT | |
| | | GTCAGCGGCTGGCGGCTGT | |
| | | TCAAGAAAATCAGTTAGGA | |
| | | CGGGGGGCGGCAACACGG | |
| | | CTCGAGCAACACTCAACCC | |
| | | TATCTCGGTCT | |
| FLAG | FLAG (affinity tag | TGCGTCCGGCGTAGAGGAT | SEQ ID NO: 13 |
| | epitope)-encoding | CCTAGTAATACGACTCACT | |
| | sequence | ATAGGGTTAACTTTAAGAA | |
| | | GGAGATATACATATGGACT | |
| | | ACAAAGACGATGACGACA | |
| | | AGGGAGGTGGTGGAAGTG | |
| | | TCAGCGGCTGGCGGCTGTT | |
| | | CAAGAAAATCAGTTAGGAC | |
| | | GGGGGGCGGCAACACGGC | |
| | | TCGAGCAACACTCAACCCT | |
| | | ATCTCGGTCT | |
| Myc | Myc (affinity tag | TGCGTCCGGCGTAGAGGAT | SEQ ID NO: 14 |
| | epitope)-encoding | CCTAGTAATACGACTCACT | |
| | sequence | ATAGGGTTAACTTTAAGAA | |
| | | GGAGATATACATATGGAAC | |
| | | AAAAACTCATCTCAGAAGA | |
| | | GGATCTGGGAGGTGGTGGA | |
| | | AGTGTCAGCGGCTGGCGGC | |
| | | TGTTCAAGAAAATCAGTTA | |
| | | GGACGGGGGGCGGCAACA | |
| | | CGGCTCGAGCAACACTCAA | |
| | | CCCTATCTCGGTCT | |
| S-tag | S-tag (affinity tag | TGCGTCCGGCGTAGAGGAT | SEQ ID NO: 15 |
| | epitope)-encoding | CCTAGTAATACGACTCACT | |
| | sequence | ATAGGGTTAACTTTAAGAA | |
| | | GGAGATATACATATGAAAG | |
| | | AAACCGCTGCTGCTAAATT | |
| | | CGAACGCCAGCACATGGAC | |
| | | AGCGGAGGTGGTGGAAGT | |
| | | GTCAGCGGCTGGCGGCTGT | |
| | | TCAAGAAAATCAGTTAGGA | |
| | | CGGGGGGCGGCAACACGG | |
| | | CTCGAGCAACACTCAACCC | |
| | | TATCTCGGTCT | |
| NNK5 Library | NNK degenerate | TGCGTCCGGCGTAGAGGAT | SEQ ID NO: 16 |
| template | pentapeptide | CCTAGTAATACGACTCACT | |
| | library-encoding | ATAGGGTTAACTTTAAGAA | |
| | sequence | GGAGATATACATATGNNKN | |
| | | NKNNKNNKNNKGGAGGTG | |
| | | GTGGAAGTGTCAGCGGCTG | |
| | | GCGGCTGTTCAAGAAAATC | |
| | | AGTTAGGACGGGGGGCGG | |
| | | CAACACGGCTCGAGCAACA | |
| | | CTCAACCCTATCTCGGTCT | |
| NNU5 Library | NNU degenerate | TGCGTCCGGCGTAGAGGAT | SEQ ID NO: 17 |
| template | pentapeptide | CCTAGTAATACGACTCACT | |
| | library-encoding | ATAGGGTTAACTTTAAGAA | |
| | sequence | GGAGATATACATATGNNTN | |
| | | NTNNTNNTNNTGGAGGTGG | |
| | | TGGAAGTGTCAGCGGCTGG | |
| | | CGGCTGTTCAAGAAAATCA | |
| | | GTTAGGACGGGGGGCGGC | |
| | | AACACGGCTCGAGCAACAC | |
| | | TCAACCCTATCTCGGTCT | |
| Azidolysine | Azidolysine (AzK) | TGCGTCCGGCGTAGAGGAT | SEQ ID NO: 18 |
| (AzK) peptide | peptide-encoding | CCTAGTAATACGACTCACT | |
| | sequence | ATAGGGTTAACTTTAAGAA | |
| | | GGAGATATACATATGGGTG | |
| | | GCCACGGTGGCCATCAGCA | |
| | | CGGCGGAGGTGGTGGAAG | |
| | | TGTCAGCGGCTGGCGGCTG | |
| | | TTCAAGAAAATCAGTTAGG | |
| | | ACGGGGGGCGGCAACACG | |
| | | GCTCGAGCAACACTCAACC | |
| | | CTATCTCGGTCT | |
| G4S linker | Glycine-Serine | GGGGS | SEQ ID NO: 19 |
| | linker | | |
| P1′ | P1 reverse | CACTCAACCC TATCTCGGT | SEQ ID NO: 20 |
| | complement | | |
| His tag | Histidine tag | HHHHHH | SEQ ID NO: 21 |

TABLE 2
DNA sequencing analysis of particles from input library
and high APC population (AzK sort NGS Results)

| Population | AzK | library |
|---|---|---|
| input | 0.09% | 99.91% |
| high APC | 0.58% | 99.42% |

Values are c total sequences matched to either epitope or the NNUs library references.

1. A composition comprising a particle comprising a magnetic core coated in a polymer gel.

2. The composition of claim 1, wherein the polymer gel comprises polyacrylamide.

3. The composition of claim 1, wherein the polymer gel comprises about 4% w/v to about 10% w/v polyacrylamide.

4. The composition of claim 1, wherein the polymer gel comprises between about 10:1 to about 40:1 acrylamide to bis acrylamide.

5. (canceled)

6. The composition of claim 1, wherein the particle further comprises an additive that inhibits phase separation, condensate formation, and/or coacervation.

7-8. (canceled)

9. The composition of claim 1, wherein about 0.001 mM to about 20 mM of the polymer gel is functionalized.

10. The composition of claim 1, wherein the particle is fluorescently labeled.

11. The composition of claim 1, wherein the polymer gel has a pore size of about 20 nm to about 200 nm.

12. The composition of claim 1, wherein the magnetic core is a magnetic bead.

13-20. (canceled)

21. A method of producing a composition comprising a particle comprising a magnetic core encapsulated in a polymer gel,
comprising emulsifying an aqueous solution comprising monomers and magnetic beads with a solution comprising a polymerization initiator to cause polymerization of the monomer and thereby producing a composition comprising a magnetic core encapsulated in the polymer gel.

22. The method of claim 21, wherein the polymer gel comprises polyacrylamide.

23. The method of claim 21, comprising emulsifying a solution comprising 4% w/v to 10% acrylamide monomer and magnetic beads with the solution comprising an initiator.

24. The method of claim 21, wherein the aqueous solution comprising monomers comprises acrylamide monomer and bis-acrylamide monomer.

25. (canceled)

26. The method of claim 21, wherein the polymerization initiator is in an oil solution.

27-29. (canceled)

30. The method of claim 24, further comprising combining the solution comprising acrylamide and bis-acrylamide monomer and magnetic beads and the solution comprising the polymerization initiator to produce a combined composition prior to emulsification.

31-37. (canceled)

38. The method of claim 21, further comprising functionalizing a portion of the gel.

39. A particle produced by the method of claim 21.

40. A library of particles produced by the method of claim 21.

41. A kit comprising a composition comprising aqueous solution comprising magnetic beads and acrylamide and a composition comprising an oil solution comprising a polymerization initiator.

42-43. (canceled)


SEQUENCE LISTING (21 sequences)

SEQ ID NO: 1 - DNA, 24 bp, synthetic construct
  source 1..24: mol_type = other DNA
agaccgagatagggttgagtgttg

SEQ ID NO: 2 - DNA, 24 bp, synthetic construct
  source 1..24: mol_type = other DNA
  misc_feature 1: note = 5' acrydite
agaccgagatagggttgagtgttg

SEQ ID NO: 3 - DNA, 20 bp, synthetic construct
  source 1..20: mol_type = other DNA
tgcgtccggcgtagaggatc

SEQ ID NO: 4 - DNA, 16 bp, synthetic construct
  source 1..16: mol_type = other DNA
  misc_feature 1: note = 5' 6-FAM
cactcaaccctatctc

SEQ ID NO: 5 - DNA, 24 bp, synthetic construct
  source 1..24: mol_type = other DNA
  misc_feature 1: note = 5' 6-FAM
cttgtcgtcatcgtctttgtagtc

SEQ ID NO: 6 - DNA, 18 bp, synthetic construct
  source 1..18: mol_type = other DNA
  misc_feature 18: note = iSp18-iSp18-iSp18-3Puro
gtgttgccgccccccgtc

SEQ ID NO: 7 - DNA, 37 bp, synthetic construct
  source 1..37: mol_type = other DNA
  modified_base 9..10: mod_base = OTHER; note = ZEN internal quencher
  misc_feature 37: note = 3' Iowa Black FQ quencher
tatatctccttcttaaagttaaccctatagtgagtcg

SEQ ID NO: 8 - DNA, 14 bp, synthetic construct
  source 1..14: mol_type = other DNA
  misc_feature 1: note = 5' phosphorylation
  modified_base 6..7: mod_base = OTHER; note = iSp9-iUniAmM-iSp (iSp9 = Int Spacer 9, which is a triethylene glycol spacer; iUniAmM = Int Uni-Link Amino Modifier, which is an amino-modifier phosphoramidite from Clontech)
gagtcatgactccc

SEQ ID NO: 9 - DNA, 26 bp, synthetic construct
  source 1..26: mol_type = other DNA
  misc_feature 9: note = iFluorT
gggccgccttcgtccttctcagcgac

SEQ ID NO: 10 - DNA, 32 bp, synthetic construct
  source 1..32: mol_type = other DNA
  misc_feature 1: note = 5' phosphorylation
ccatgtcgctgagaaggacgaaggcggcccgg

SEQ ID NO: 11 - DNA, 215 bp, synthetic construct
  source 1..215: mol_type = other DNA
tgcgtccggcgtagaggatcctagtaatacgactcactatagggttaactttaagaaggagatatacatatgggtaagcctatccctaaccctctcctcggtctcgattctacgggaggtggtggaagtgtcagcggctggcggctgttcaagaaaatcagttaggacggggggcggcaacacggctcgagcaacactcaaccctatctcggtct

SEQ ID NO: 12 - DNA, 200 bp, synthetic construct
  source 1..200: mol_type = other DNA
tgcgtccggcgtagaggatcctagtaatacgactcactatagggttaactttaagaaggagatatacatatgtacccatacgatgttccagattacgctggaggtggtggaagtgtcagcggctggcggctgttcaagaaaatcagttaggacggggggcggcaacacggctcgagcaacactcaaccctatctcggtct

SEQ ID NO: 13 - DNA, 197 bp, synthetic construct
  source 1..197: mol_type = other DNA
tgcgtccggcgtagaggatcctagtaatacgactcactatagggttaactttaagaaggagatatacatatggactacaaagacgatgacgacaagggaggtggtggaagtgtcagcggctggcggctgttcaagaaaatcagttaggacggggggcggcaacacggctcgagcaacactcaaccctatctcggtct

SEQ ID NO: 14 - DNA, 203 bp, synthetic construct
  source 1..203: mol_type = other DNA
tgcgtccggcgtagaggatcctagtaatacgactcactatagggttaactttaagaaggagatatacatatggaacaaaaactcatctcagaagaggatctgggaggtggtggaagtgtcagcggctggcggctgttcaagaaaatcagttaggacggggggcggcaacacggctcgagcaacactcaaccctatctcggtct

SEQ ID NO: 15 - DNA, 218 bp, synthetic construct
  source 1..218: mol_type = other DNA
tgcgtccggcgtagaggatcctagtaatacgactcactatagggttaactttaagaaggagatatacatatgaaagaaaccgctgctgctaaattcgaacgccagcacatggacagcggaggtggtggaagtgtcagcggctggcggctgttcaagaaaatcagttaggacggggggcggcaacacggctcgagcaacactcaaccctatctcggtct

SEQ ID NO: 16 - DNA, 188 bp, synthetic construct
  source 1..188: mol_type = other DNA
tgcgtccggcgtagaggatcctagtaatacgactcactatagggttaactttaagaaggagatatacatatgnnknnknnknnknnkggaggtggtggaagtgtcagcggctggcggctgttcaagaaaatcagttaggacggggggcggcaacacggctcgagcaacactcaaccctatctcggtct

SEQ ID NO: 17 - DNA, 188 bp, synthetic construct
  source 1..188: mol_type = other DNA
tgcgtccggcgtagaggatcctagtaatacgactcactatagggttaactttaagaaggagatatacatatgnntnntnntnntnntggaggtggtggaagtgtcagcggctggcggctgttcaagaaaatcagttaggacggggggcggcaacacggctcgagcaacactcaaccctatctcggtct

SEQ ID NO: 18 - DNA, 200 bp, synthetic construct
  source 1..200: mol_type = other DNA
tgcgtccggcgtagaggatcctagtaatacgactcactatagggttaactttaagaaggagatatacatatgggtggccacggtggccatcagcacggcggaggtggtggaagtgtcagcggctggcggctgttcaagaaaatcagttaggacggggggcggcaacacggctcgagcaacactcaaccctatctcggtct

SEQ ID NO: 19 - protein, 5 aa, synthetic construct
GGGGS

SEQ ID NO: 20 - DNA, 19 bp, synthetic construct
  source 1..19: mol_type = other DNA
  misc_feature 1: note = 5' 6-FAM
cactcaaccctatctcggt

SEQ ID NO: 21 - protein, 6 aa, synthetic construct
HHHHHH